SEQUENCE LISTING (23 sequences)

SEQ ID NO: 1 - protein, 349 aa, Yarrowia lipolytica
MVLSTVIGEWFSRVLFGTVAPSPLTATAPISQDFYDTALTFSHLSNVAYCINTPLESLKSDFSCGVACSHFPNMELVEEFGGEFFETSITGFLSIDHVKKEKYVVYRGTYDIGDVYTDIQLSQSPFLVTPSALGSTANLCEGCTIHDGWNKAYNETMGIIGDKLADHVNSNPDYRLVVTGHSLGAAIAVLSATSLKVNGQDPYLYTYGQPRIGNANFANFVSKQWFGEGDGLSMDSDRRYFRLTHWNDLFVGFPAFKDYVHSVGEIYIDYFTVQPPLNKVFSCAGPESMSCYRKDFNALARLDIVKNHLAYFDWISLCTLNIGRRDLERGRKFEGTWLYGGLANGSTIF

SEQ ID NO: 2 - protein, 307 aa, Yarrowia lipolytica
AVLQKRVYTSTETSHIDQESYNFFEKYARLANIGYCVGPGTKIFKPFNCGLQCAHFPNVELIEEFHDPRLIFDVSGYLAVDHASKQIYLVIRGTHSLEDVITDIRIMQAPLTNFDLAANISSTATCDDCLVHNGFIQSYNNTYNQIGPKLDSVIEQYPDYQIAVTGHSLGGAAALLFGINLKVNDHDPLVVTLGQPIVGNAGFANWVDKLFFGQENPDVSKVSKDRKLYRITHRGDIVPQVPFWDGYQHCSGEVFIDWPLIHPPLSNVVMCQGQSNKQCSAGNTLLQQVNVIGNHLQYFVTEGVCGI

SEQ ID NO: 3 - protein, 347 aa, Yarrowia lipolytica
QTAPITQETYDFVLKYGWLSNVAYCVRAPGPFALQSDFTCGNSCAHFPDVTLDYQFGGNFFSTSVTGFLAHDHTKKEKYIVFRGTFSIADAITDIQTIQQPYMTSIPPLNTTDINSTNPSASINCPGCQVHDGFQKAYRETMVNVQDRLVDFLGNNTDYKLIVTGHSLGAVTALFMGINLKNLGYDPTLINYGQPRLGNKAFADYVDALFFKQGDDGLTINPERRMYRVTHWNDFFVGWPAGYSHTLGEVYISDPTGINAPIEDVYSCAGPENNQCHHGSFNLLERLNILKNHCGYLNWIFYCAINVDKREMMIDPPRVGKRVEHWSGKFSDVESTEGLMYEAIYPM

SEQ ID NO: 4 - protein, 279 aa, Aspergillus niger
APAPAPMQRRDISSTVLDNIDLFAQYSAAAYCSSNIESTGTTLTCDVGNCPLVEAAGATTIDEFDDSSSYGDPTGFIAVDPTNELIVLSFRGSSDLSNWIADLDFGLTSVSSICDGCEMHKGFYEAWEVIADTITSKVEAAVSSYPDYTLVFTGHSYGAALAAVAATVLRNAGYTLDLYNFGQPRIGNLALADYITDQNMGSNYRVTHTDDIVPKLPPELLGYHHFSPEYWITSGNDVTVTTSDVTEVVGVDSTAGNDGTLLDSTTAHRWYTIYISECS

SEQ ID NO: 5 - protein, 261 aa, Aspergillus niger
VPMLQSRATSDPAEWTELHRAAQLSSAAYTGCTGSAFDVTITKQINELVTDTQGFIGYSTEKKRITVAMRGSTSATDIANDVDTTLVEPTLSGVNFPSGAKMMHGIYSPWSSVHDDVISEVKSLVEQYPDYTIESTGHSLGGSLTYISYIALAQNFPGKTIISNALAAYPIGNEAFANFGASQNGTLNRGNNADDGVPNMYVMWPWDFVHYGTEYYSSGTQASTVKCSGERDTSCSAGNGQVGVTAGHFSNFGIAMGMAGC

SEQ ID NO: 6 - protein, 333 aa, Mucor javanicus
APASNDATIADVSSTFTLPPIIKDRVAFSNDIADTDYFNLTKRASETVGGNTMDLPSNAPALPAVPKAGDVVIATAAQIAEYKKYAALASTAYCRSVVPLNLWTCVNCLRFAPDGKLIKTFSSVISDTNGFVLRSDAQKTIYVVFRGTNSIRSAITDLVFTLISYPPVSGARVHTGFYASYQAVVSDYFPVVQSQLTSYPSYKVVVTGHSLGGAHALLAGMDLYQREKRLTASNLFIHTAGCPRVGNPTFANYVASTGITFTRSVNQRDIVPHVPPTYAGYLHPGVEVWARTSSTVQICTSNTESNMCSNSIEPFTSFTDHLSYYGITEGVCI

SEQ ID NO: 7 - protein, 287 aa, Penicillium camemberti
SPVQLARRAISSELLERFTLFSQFATLSACDQNINHTGQSLTCDYGTCGLVAADNTTVINAFHSDNGPTGYIALDHTRQLIVLTFRGTVSKSDGDTDLDIVLTSIDDVCTGCKAHHGFWVYWSAVASQATTQLQDATSAYPSYRLSVVGHSLGGGIAALAGTVLRTQGFTLDIWTFGGPKPGNLKLAEFITNQQPPNSIYRATHTTDPIPKVPLNLPFLDWSQPSPEYWITQETGVQVTTDGVEYIEGINSKAGNAGSDRDLRWPNPEHGWYFGNMSVCASPSDASS

SEQ ID NO: 8 - protein, 275 aa, Penicillium decumbens
APSQLVPRAVSSGTLDQLTLFAQYSAASYCANNVNSPGDAITCSGGYCPKLQSAGVKSLFEFDDSTEFGDVAGFLSVDTANKLLVLSFRGSRTISNWIANLDFGQADASSLCIGCKAHSGFLKAWTVVSDDVMPPLVSAMAKYPGFRLVLTGHSFGGAVAALGATALRKAGYKLDLYTYGQPRVGNTALATYMTNQGSMYRVTHSNDIVPKLPPPLLGYTHASPEYWITSGDNVAVTTKDITQVNGIGSKDGNAGSNGDSIPAHNWYIVNIDGCK

SEQ ID NO: 9 - protein, 271 aa, Penicillium decumbens
APQKRSVSSTVLAQLSRYAQWSAAAYCSGNTSGANQVVSCSANNCPDVQASGATMLYEFDSTNTYGDAAGFLAADTTQQQIILSFRGSRTTSNWIANLDTELTSSTLCSGCEVHQGFWLDYQTVAATLKAQIDAALNTYPGYSLVVTGHSLGGALAMLAGLDLNSQGYAPTIYTYGQPRVGNLALAQYITNVGNQWRVTHADDAVPKLPPRLFGFSHASPEYWITSGDNVAVTTGDVTVVTGVNSLGGNDGTLTASVSAHNWYLVDIDACK

SEQ ID NO: 10 - protein, 269 aa, Pleurotus ostreatus
APALPLEVRDSGVSQAVYDDLVVYAKYSSAVYQPFCPRPLGSWMIKAFDTKGTQGFVARDDARKEIIVAFRGSFEIVDILIDIQIILTPLSTPGVSNVGSARVHTGFQKAYNFVIDEVQSLMKSQIDAYPSYKLVVTGHSLGGAVATFAALALKSKYPSKSLRLFTYGAPRVGDAAFASLVESRLGINNIYRGVHTWDGVPTLLGRWLGYRHYGTEYWQHKDPSKPENVRKCNGGEDTSCSNSIISTGINPPHAFQFGQVMAINPLLCF

SEQ ID NO: 11 - protein, 366 aa, Yarrowia lipolytica
MVSFGARIKDFFSVLLFGAASTSSSTKTALVSQGFYDAALDFSHLSNIAYCVNAPITPLKSDFSCGQSCVHFPDIELVHIFGGDFFSTSITGYLALDHVKKEKYVVFRGTFSIADAITDIQFQQSSFLVNVPALNTFTANDTAPEAQIDCKQCKIHDGFSKAFTETWHNIGDLLEQHLDSYPDYQLYVTGHSLGAAMALLGATSIKLRGYDPILINYGQPRVGNKAFADYISALWFGNGDGLEINQQRRLYRMTHWNDVFVGLPNWDGYTHSNGEVYIKGKYVNPPLKDVFSCAGGENSKCYRSEFNLLAQINLLQNHLCYIDYIGFCALNVGRRELNDLPHYNGPYKYGHKTEEQFIAEGLELSN

SEQ ID NO: 12 - DNA, 1047 bp, Yarrowia lipolytica
  source 1..1047: mol_type = genomic DNA
atggttttgtctactgttattggtgaatggttctctagagttttgtttggtactgttgctccatctcctttgactgctactgctccaatctctcaagatttctacgatactgctttgactttttctcatttgtctaacgttgcttactgtatcaacactccattggaatctttgaagtctgatttctcttgtggtgttgcttgttctcactttcctaacatggagttggttgaagagttcggtggtgaatttttcgagacttctattactggtttcttgtctatcgatcatgttaagaaagagaagtacgttgtttacagaggtacttacgatatcggagatgtttacactgatatccaattgtctcaatctccattcttggttactccttctgctttgggttctactgctaatttgtgtgaaggttgtactattcacgatggttggaacaaggcttataatgagactatgggtattattggagataaattggctgatcatgttaactctaatcctgattacagattggttgttactggtcactctttgggtgctgctattgctgttttgtctgctacttctttgaaggttaacggtcaagatccatacttgtatacttacggtcaacctagaatcggtaacgctaacttcgctaacttcgtttctaagcaatggtttggtgaaggagatggtttgtctatggattctgatagaagatacttcagattgactcattggaacgatttgtttgttggtttcccagcttttaaggattacgttcactctgttggtgaaatctacatcgattacttcactgttcaaccacctttgaacaaggttttctcttgtgctggtcctgagtctatgtcttgttacagaaaggatttcaacgctttggctagattggatatcgttaaaaatcatttggcttacttcgattggatttctttgtgtactttgaacattggtagaagagatttggaaagaggtagaaagtttgagggtacttggttgtatggtggtttggctaatggttctactattttc

SEQ ID NO: 13 - DNA, 921 bp, Yarrowia lipolytica
  source 1..921: mol_type = genomic DNA
gctgttttgcaaaagagagtttacacttctactgaaacttctcatatcgatcaagaatcttacaactttttcgagaagtatgctagattggctaatattggttattgtgttggtccaggtactaagatttttaagcctttcaactgtggtttgcaatgtgctcatttcccaaacgttgaattgatcgaagagtttcacgatcctagattgattttcgatgtttctggttacttggctgttgatcatgcttctaagcaaatctacttggttattagaggtactcactctttggaggatgttatcactgatatcagaatcatgcaagctccattgactaactttgatttggctgctaatatttcttctactgctacttgtgatgattgtttggttcacaacggtttcattcaatcttacaacaacacttacaatcaaattggtccaaagttggattctgttattgagcaataccctgattatcaaattgctgttactggtcattctttgggtggtgctgctgctttgttgttcggtattaacttgaaggttaatgatcacgatccattggttgttactttgggtcaacctattgttggtaacgctggtttcgctaattgggttgataagttgtttttcggtcaagaaaacccagatgtttctaaggtttctaaggatagaaagttgtacagaatcactcatagaggagatattgttccacaagttcctttttgggatggttatcaacattgttctggagaggttttcattgattggcctttgattcacccacctttgtctaatgttgttatgtgtcaaggtcaatctaacaaacaatgttctgctggtaacactttgttgcaacaagttaacgttattggtaatcacttgcaatacttcgttactgaaggtgtttgtggtatt

SEQ ID NO: 14 - DNA, 1041 bp, Yarrowia lipolytica
  source 1..1041: mol_type = genomic DNA
caaactgctccaatcactcaagaaacttacgatttcgttttgaagtacggttggttgtctaacgttgcttactgtgttagagctccaggtccttttgctttgcaatctgatttcacttgtggtaactcttgtgctcatttccctgatgttactttggattaccaattcggtggtaactttttctctacttctgttactggtttcttggctcacgatcacactaagaaagaaaagtacatcgtttttagaggtactttctctatcgctgatgctattactgatatccaaactatccaacaaccatacatgacttctattccacctttgaacactactgatatcaactctactaatccatctgcttctattaattgtcctggttgtcaagttcacgatggttttcaaaaagcttacagagagactatggttaacgttcaagatagattggttgatttcttgggtaacaacactgattacaagttgatcgttactggtcactctttgggtgctgttactgctttgtttatgggtattaacttgaaaaatttgggttacgatccaactttgattaactatggtcaacctagattgggtaataaggctttcgctgattacgttgatgctttgtttttcaaacaaggagatgatggtttgactattaacccagaaagaagaatgtacagagttactcattggaacgatttctttgttggttggcctgctggttactctcacactttgggtgaagtttatatttctgacccaactggtattaacgctcctattgaagatgtttactcttgtgctggtccagagaacaatcaatgtcatcacggttcttttaatttgttggaaagattgaacatcttgaagaatcattgtggttacttgaactggattttctattgtgctattaatgttgataagagagaaatgatgattgatccacctagagttggtaaaagagttgagcactggtctggtaaattttctgatgttgaatctactgagggtttgatgtacgaggctatctaccctatg

SEQ ID NO: 15 - DNA, 837 bp, Aspergillus niger
  source 1..837: mol_type = genomic DNA
gctccagctcctgctccaatgcaaagaagagatatctcttctactgttttggataacatcgatttgttcgctcaatactctgctgctgcttattgttcttctaacattgaatctactggtactactttgacttgtgatgttggtaattgtcctttggttgaagctgctggtgctactactatcgatgagttcgatgattcttcttcttacggagatcctactggtttcattgctgttgatccaactaacgagttgattgttttgtcttttagaggttcttctgatttgtctaattggattgctgatttggatttcggtttgacttctgtttcttctatttgtgatggttgtgaaatgcataagggtttttacgaagcttgggaggttattgctgatactatcacttctaaggttgaggctgctgtttcttcttaccctgattatactttggttttcactggtcactcttatggtgctgctttggctgctgttgctgctactgttttgagaaatgctggttacactttggatttgtataactttggtcaaccaagaattggtaatttggctttggctgattacatcactgatcaaaacatgggttctaactacagagttactcatactgatgatattgttcctaagttgccacctgaattgttgggttaccatcacttttctccagagtattggattacttctggtaacgatgttactgttactacttctgatgttactgaagttgttggtgttgattctactgctggtaatgatggtactttgttggattctactactgctcacagatggtacactatctacatttctgagtgttct

SEQ ID NO: 16 - DNA, 783 bp, Aspergillus niger
  source 1..783: mol_type = genomic DNA
gttccaatgttgcaatctagagctacttctgatcctgctgaatggactgagttgcatagagctgctcaattgtcttctgctgcttacactggttgtactggttctgctttcgatgttactatcactaagcaaattaacgaattggttactgatactcaaggtttcattggttactctactgagaagaaaagaatcactgttgctatgagaggttctacttctgctactgatattgctaacgatgttgatactactttggttgaaccaactttgtctggtgttaattttccttctggtgctaagatgatgcatggtatctactctccatggtcttctgttcacgatgatgttatctctgaggttaagtctttggttgagcaataccctgattatactattgaatctactggtcactctttgggtggttctttgacttacatctcttacatcgctttggctcaaaacttcccaggtaaaactatcatctctaacgctttggctgcttatcctattggtaacgaggcttttgctaatttcggtgcttctcaaaacggtactttgaatcgtggtaacaatgctgatgatggtgttccaaatatgtacgttatgtggccttgggatttcgttcattacggtactgaatactactcttctggtactcaagcttctactgttaaatgttctggagagagagatacttcttgttctgctggtaacggtcaagttggtgttactgctggtcacttttctaatttcggtattgctatgggtatggctggttgt

SEQ ID NO: 17 - DNA, 999 bp, Mucor javanicus
  source 1..999: mol_type = genomic DNA
gctcctgcttctaatgatgctactattgctgatgtttcttctactttcactttgccacctattattaaggatagagttgctttttctaacgatatcgctgatactgattacttcaacttgactaagagagcttctgaaactgttggtggtaatactatggatttgccatctaacgctccagctttgcctgctgttccaaaggctggagatgttgttattgctactgctgctcaaattgctgagtacaagaaatatgctgctttggcttctactgcttactgtagatctgttgttcctttgaatttgtggacttgtgttaactgtttgagatttgctccagatggtaaattgattaaaactttctcttctgttatttctgatactaacggtttcgttttgagatctgatgctcaaaagactatctacgttgttttcagaggtactaactctatcagatctgctatcactgatttggtttttactttgatctcttacccacctgtttctggtgctagagttcatactggtttctacgcttcttatcaagctgttgtttctgattactttcctgttgttcaatctcaattgacttcttacccatcttataaggttgttgttactggtcattctttgggtggtgctcacgctttgttggctggtatggatttgtaccaaagagaaaagagattgactgcttctaatttgtttattcacactgctggttgtcctagagttggtaatccaactttcgctaactacgttgcttctactggtatcacttttactagatctgttaaccaaagagatattgttcctcatgttccacctacttacgctggttatttgcacccaggtgttgaagtttgggctagaacttcttctactgttcaaatctgtacttctaacactgaatctaacatgtgttctaactctatcgagccttttacttctttcactgatcatttgtcttactatggtattactgagggtgtttgtatt

SEQ ID NO: 18 - DNA, 861 bp, Penicillium camemberti
  source 1..861: mol_type = genomic DNA
tctccagttcaattggctagaagagctatttcttctgaattgttggagagattcactttgttctctcaatttgctactttgtctgcttgtgatcaaaacatcaaccatactggtcaatctttgacttgtgattacggtacttgtggtttggttgctgctgataacactactgttattaatgctttccattctgataacggtcctactggttatattgctttggatcacactagacaattgatcgttttgacttttagaggtactgtttctaagtctgatggagatactgatttggatatcgttttgacttctatcgatgatgtttgtactggttgtaaagctcatcacggtttctgggtttactggtctgctgttgcttctcaagctactactcaattgcaagatgctacttctgcttacccatcttatagattgtctgttgttggtcattctttgggtggtggtattgctgctttggctggtactgttttgagaactcaaggtttcactttggatatttggacttttggtggtccaaagcctggtaacttgaagttggctgaattcattactaaccaacaaccacctaattctatctacagagctactcacactactgatccaattcctaaggttccattgaatttgccatttttggattggtctcaaccatctcctgaatactggattactcaagagactggtgttcaagttactactgatggtgttgaatacatcgagggtattaactctaaagctggtaatgctggttctgatagagatttgagatggccaaaccctgagcacggttggtattttggtaatatgtctgtttgtgcttctccttctgatgcttcttct

SEQ ID NO: 19 - DNA, 825 bp, Penicillium decumbens
  source 1..825: mol_type = genomic DNA
gctccttctcaattggttccaagagctgtttcttctggtactttggatcaattgactttgtttgctcaatactctgctgcttcttattgtgctaacaatgttaactctcctggagatgctattacttgttctggtggttactgtccaaagttgcaatctgctggtgttaagtctttgttcgaattcgatgattctactgagtttggagatgttgctggtttcttgtctgttgatactgctaataagttgttggttttgtcttttagaggttctagaactatctctaactggatcgctaatttggatttcggtcaagctgatgcttcttctttgtgtattggttgtaaggctcattctggtttcttgaaagcttggactgttgtttctgatgatgttatgccacctttggtttctgctatggctaaataccctggttttagattggttttgactggtcactctttcggtggtgctgttgctgctttgggtgctactgctttgagaaaggctggttacaagttggatttgtacacttacggtcaaccaagagttggtaacactgctttggctacttacatgactaatcaaggttctatgtacagagttactcattctaacgatatcgttcctaagttgccacctccattgttgggttacactcacgcttctccagaatattggattacttctggagataacgttgctgttactactaaggatatcactcaagttaacggtatcggttctaaagatggtaacgctggttctaatggagattctattcctgctcataactggtatattgttaatattgatggttgtaaa

SEQ ID NO: 20 - DNA, 813 bp, Penicillium decumbens
  source 1..813: mol_type = genomic DNA
gctccacaaaagagatctgtttcttctactgttttggctcaattgtctagatacgctcaatggtctgctgctgcttattgttctggtaacacttctggtgctaatcaagttgtttcttgttctgctaacaattgtcctgatgttcaagcttctggtgctactatgttgtacgaattcgattctactaacacttacggagatgctgctggtttcttggctgctgatactactcaacaacaaatcatcttgtcttttagaggttctagaactacttctaactggattgctaatttggatactgaattgacttcttctactttgtgttctggttgtgaggttcatcaaggtttctggttggattaccaaactgttgctgctactttgaaagctcaaattgatgctgctttgaatacttacccaggttattctttggttgttactggtcactctttgggtggtgctttggctatgttggctggtttggatttgaactctcaaggttacgctccaactatctacacttatggtcaacctagagttggtaatttggctttggctcaatacatcactaacgttggtaaccaatggagagttactcatgctgatgatgctgttccaaagttgccacctagattgtttggtttctctcacgcttctcctgagtactggattacttctggagataacgttgctgttactactggagatgttactgttgttactggtgttaactctttgggtggtaatgatggtactttgactgcttctgtttctgctcataactggtatttggttgatatcgatgcttgtaaa

SEQ ID NO: 21 - DNA, 807 bp, Pleurotus ostreatus
  source 1..807: mol_type = genomic DNA
gctccagctttgcctttggaagttagagattctggtgtttctcaagctgtttacgatgatttggttgtttacgctaagtattcttctgctgtttatcaaccattttgtccaagacctttgggttcttggatgattaaggcttttgatactaaaggtactcaaggtttcgttgctagagatgatgctagaaaggaaatcatcgttgcttttagaggttctttcgagattgttgatatcttgatcgatatccaaatcatcttgactccattgtctactcctggtgtttctaacgttggttctgctagagttcatactggtttccaaaaggcttacaacttcgttatcgatgaggttcaatctttgatgaagtctcaaatcgatgcttacccttcttacaagttggttgttactggtcactctttgggtggtgctgttgctactttcgctgctttggctttgaagtctaagtacccatctaagtctttgagattgtttacttacggtgctcctagagttggagatgctgctttcgcttctttggttgaatctagattgggtattaacaacatctacagaggtgttcatacttgggatggtgttccaactttgttgggtagatggttgggttacagacattatggtactgagtattggcaacacaaggatccatctaaacctgaaaacgttagaaagtgtaatggtggagaggatacttcttgttctaactctatcatctctactggtattaatccacctcacgcttttcaattcggtcaagttatggctattaaccctttgttgtgtttt

SEQ ID NO: 22 - DNA, 1098 bp, Yarrowia lipolytica
  source 1..1098: mol_type = genomic DNA
atggtttctttcggtgctagaattaaggatttcttttctgttttgttgtttggtgctgcttctacttcttcttctactaaaactgctttggtttctcaaggtttttatgatgctgctttggatttctctcatttgtctaacatcgcttactgtgttaacgctccaattactcctttgaagtctgatttttcttgtggtcaatcttgtgttcatttcccagatattgaattggttcacatttttggtggagatttcttttctacttctatcactggttatttggctttggatcacgttaagaaagagaagtacgttgtttttagaggtactttctctatcgctgatgctatcactgatatccaattccaacaatcttctttcttggttaacgttccagctttgaatactttcactgctaacgatactgctcctgaagctcaaatcgattgtaagcagtgtaagattcacgatggtttttctaaggctttcactgaaacttggcataacattggagatttgttggagcaacacttggattcttacccagattatcaattgtacgttactggtcactctttgggtgctgctatggctttgttgggtgctacttctattaagttgagaggttacgatccaatcttgattaattacggtcaacctagagttggtaacaaagctttcgctgattatatttctgctttgtggtttggtaatggagatggtttggaaattaaccaacaaagaagattgtacagaatgactcattggaacgatgttttcgttggtttgcctaactgggatggttacactcactctaacggagaggtttacatcaagggtaaatacgttaacccacctttgaaggatgttttctcttgtgctggtggtgaaaactctaagtgttacagatctgagtttaatttgttggctcaaattaacttgttgcaaaaccatttgtgttacatcgattacatcggtttctgtgctttgaacgttggtagaagagagttgaatgatttgccacattacaacggtccttacaagtatggtcacaaaactgaagagcaattcattgctgaaggtttggagttgtctaat

SEQ ID NO: 23 - DNA, 1047 bp, Synthetic construct
  misc_feature 1..1047: note = >SEQ ID NO: 23
  source 1..1047: mol_type = other DNA
atggtcctgtccactgttatcggagaatggttttcaagagttttgtttggtactgtcgccccatcaccactcaccgccacagcccccatttctcaggacttctacgataccgctctcacgttctctcacctatctaatgttgcttactgtatcaacacccctcttgagtccctcaagtccgacttttcctgcggtgtcgcatgttctcacttcccaaacatggaacttgttgaagagttcggtggagaatttttcgaaacttctattactggttttctatctatcgaccatgtcaaaaaggaaaagtacgttgtgtacagaggcacctatgacattggtgacgtttatactgacattcaattaagccaatccccattcttggttaccccttctgccctgggttctacagcaaatctgtgtgagggttgtactatccacgatggttggaacaaagcatacaacgaaaccatgggtattatcggagacaagctcgctgaccacgttaactccaacccagattacagattggtcgtaactggacattcacttggtgctgctattgctgtactatctgcaacctctcttaaggtcaatggccaagatccttacctttacacttacggtcagcctcgtattggtaatgctaacttcgcaaactttgtgagtaagcaatggttcggtgagggtgacggtctatctatggactcggacagacgttatttcagactgactcactggaacgacctctttgttggtttccctgctttcaaggactacgtgcactctgtcggagagatttacatagactatttcaccgttcaaccaccactcaacaaagttttctcttgtgctgggcctgagtctatgtcctgttacagaaaggacttcaacgcccttgctagacttgatattgtgaaaaaccacctagcttacttcgattggatctctctgtgcaccctaaacatcggcagacgtgatctggagaggggtagaaagttcgagggtacttggctctacggtggactggctaacggatccactatcttc

(19) United States
(12) Patent Application Publication
(10) Pub. No.: US 2026/0344690 A1
(43) Pub. Date: Nov. 5, 2026

(54) Lipases, Compositions, Methods and Uses Thereof

(71) Applicant: CHR. HANSEN A/S, Hoersholm (DK)

(72) Inventors: Christian JAECKEL, Hoersholm (DK); Hans RAJ, Hoersholm (DK)

(73) Assignee: CHR. HANSEN A/S, Hoersholm (DK)

(21) Appl. No.: 19/391,844

(22) Filed: Nov. 17, 2025

Related U.S. Application Data
(62) Division of application No. 17/798,153, filed on Feb. 26, 2021, now Pat. No. 12,495,812.

(30) Foreign Application Priority Data
Feb. 28, 2020 (EP) 20160150.7
Aug. 31, 2020 (EP) 20193597.0

(51) Int. Cl. A23C 19/032; A23C 9/12; A23C 13/16; A23C 15/12; A23C 20/00; C12N 9/20

(52) U.S. Cl. CPC A23C 19/0328; A23C 9/1216; A23C 13/16; A23C 15/123; A23C 20/00; C12N 9/20; C12Y 301/01003

(57) ABSTRACT

The present invention relates to a wild-type, non-engineered, microbial lipolytic enzyme with a higher specificity for short-chain fatty acids than for medium to long fatty acids, specially long fatty acids. This invention further relates to a process for preparing a food product, and to the use of the wild-type, non-engineered, microbial lipolytic enzyme.

### CROSS-REFERENCE TO RELATED APPLICATIONS

This application is a divisional of U.S. application Ser. No. 17/798,153 filed on Aug. 8, 2022 (now pending) which is a 35 U.S.C. 371 national application of international application no. PCT/EP2021/054825 filed Feb. 26, 2021 which claims priority or the benefit under 35 U.S.C. 119 of European application nos. 20193597.0 and 20160150.7 filed Aug. 31, 2020 and Feb. 28, 2020, the contents of which are fully incorporated herein by reference.

### SEQUENCE LISTING

This application contains a Sequence Listing in computer readable form, which is incorporated herein by reference The contents of the electronic sequence listing created on Nov. 13, 2025, named SQ.xml and is 35,651 bytes in size, is hereby incorporated by reference in its entirety.

### TECHNICAL FIELD

The present invention relates to a wild-type, non-engineered, microbial lipolytic enzyme with a high specificity for short-chain fatty acids, preferably with a higher specificity for short chain fatty acids than for medium to long chain fatty acids, preferably when compared to a known microbial lipolytic enzyme, a nucleic acid encoding said lipolytic enzyme, a vector, a host cell and/or a composition comprising the lipolytic enzyme. This invention also relates to a process for preparing a food product, such as a dairy product, wherein the lipolytic enzyme is used.

This invention is suitable for use in the food industry, preferably in the dairy industry, more preferably in the cheese industry leading, for example, to flavor enhancement or shortening of the ripening times for ripened cheeses.

### BACKGROUND

Lipolytic enzyme also called lipases (EC 3.1.1.3) are a class of hydrolases that hydrolyze ester bonds of lipids of triglycerides generating free fatty acids. This class of enzymes is responsible, for example, for the lipolysis of milk fat and/or other fat and subsequent release of short (C₄-to C₆-fatty acids), medium and/or long (C₈- and higher) chain fatty acids.

Each lipolytic enzyme has its own release profile of free fatty acids from milk fat and/or other fat. This profile may range from short (C₄- to C₆-fatty acids), to medium and/or long (C₈- and higher) chain fatty acids. The mixture of short (C₄- to C₆-fatty acids), medium and/or long (C₈- and higher) chain fatty acids obtained is dependent, at least, on the lipolytic enzyme and on the lipolytic substrate. The mixture of short (C₄- to C₆-fatty acids), medium and/or long (C₈- and higher) chain fatty acids obtained is responsible for modulating the flavor of a given final product. For example, a mixture having a higher amount or concentration of short chain fatty acids, such as a higher amount or concentration of butyric acid (C₄-fatty acid), than an amount or concentration of medium and/or long chain fatty acids leads to the development of a preferred rancid cheese flavor, in particular during ripening, and has been used for flavor generation in dairy products, for example in various cheese types. In contrast, a mixture having a higher amount or concentration of medium and/or long chain fatty acids, specially a mixture having a higher amount or concentration of long chain fatty acids (C₁₆- and higher), than an amount or concentration of short chain fatty acids gives rise to off flavors.

Lipolytic enzymes or lipases can be of animal origin or non-animal origin. Lipases from animal origin (kid goat, calf or lamb) preferably cleave short chain fatty acids (C₄- to C₆-fatty acids) from milk fat and/or other fat rather than long chain fatty acids. These short chain fatty acids, in particular C₄-fatty acids, are responsible for the formation of the preferred rancid taste rather than the soapy taste produced by the medium to long chain fatty acids. To lesser extent they also release medium chain fatty acids, which add animal specific taste to the cheese. Nevertheless, lipases from animal origin cannot be used in vegetarian and/or kosher products, which is a highly demanded market. On the other hand, lipases from non-animal origin, such as from microbial origin, fully fulfill the vegetarian and/or kosher requirements, however microbial lipases are known for being non-specific lipases mainly releasing medium to long chain fatty acids (C₈- and higher) from milk fat and/or other fat, thereby giving cheese an extremely unpleasant soapy taste.

Therefore, there is a driver in the food industry, especially in the cheese industry, to look for a new source of a microbial wild-type, non-engineered, lipase presenting a higher specificity towards short chain fatty acids, such as C₄-fatty acids and/or C₆-fatty acids, than towards medium or long chain fatty acids, and/or to look for a new source of a microbial wild-type, non-engineered, lipase presenting a higher specificity towards short chain fatty acids, such as C₄-fatty acids and/or C₆-fatty acids, and additionally a specificity towards medium chain fatty acids, such as C₈- or C₁₀-fatty acids, than towards long chain fatty acids.

Several attempts to replace animal lipases are known in the prior art. These attempts include the use of microbial lipases, recombinant lipases and/or genetic engineered lipases. However, these attempts have failed to deliver a microbial wild-type lipase with a higher specificity towards short chain fatty acids, preferably towards C₄-fatty acids, and a desirable flavor development in food products such as in cheese.

Microbial lipases can be obtained, for example, from Aspergillus, Rhizopus, and Mucor. The effect of these fungal lipases for developing rancidity in fats with respect to Aspergillus is disclosed by Iwai et al. in J. Gen. Appl. Microbiol. 10, 13-22 (1964), U.S. Pat. No. 4,726,954 or U.S. Pat. No. 4,636,468; with respect to Rhizopus is described by Oi et al. in Agr. Biol. Chem. 33, 729-38 (1969); and with respect to Mucor is set forth by Somkuti et al. in Appl. Microbiol. 16, 617-19 (1968). All the mentioned lipases have a higher specificity towards medium to long chain fatty acids, such as Aspergillus or Rhizopus, or towards medium chain fatty acids, as it is the case of Mucor, than towards short chain fatty acids, such as C₄- to C₆-fatty acids.

Microbial lipases can also be obtained from Yarrowia, such as Yarrowia (Y.) lipolytica. Several documents disclose the preference of lipases from Yarrowia towards medium to long chain fatty acids. For example, Kamoun et al. 2015 describes a higher specificity for trioctanoin than for tributyrin at pH 7.5 for Lip8; Aloulou et al. 2007 discloses a higher specificity for trioctanoin than for tributyrin at pH 6 for Lip2; Sheng, J., et al. 2012 mentions the LipY has the highest activity on C₈-C₁₂ fatty acids.

US2005059130 (or EP 2 290 059) also describes microbial lipases derived from Humicola lanuginosa, Fusarium oxysporum or Rhizomucor miehei (examples 10, 11 or 12, respectively), wherein the substrate specificity of the wild type enzymes was modified by making alterations to the amino acid sequences of said lipases and as such obtain variants with increased specificity for short-chain fatty acids. EP 3 081 644 discloses a modified lipase from Candida cylindracea also having higher specificity towards short-chain fatty acids. However, none of these microbial engineered lipases is a naturally occurring lipase with a higher specificity for short chain fatty acids than for medium to long chain fatty acids.

The foregoing illustrates the difficulty in obtaining a microbial wild-type lipase with higher specificity towards short chain fatty acids, specially towards C₄-fatty acids, and a lower specificity towards medium and/or long chain fatty acids, specially a lower specificity towards long chain fatty acids, leading to a reduction in soapiness and an increase in butyric flavors in a food product, such as a dairy product or cheese, and therefore able to replace the microbial lipases currently available.

### SUMMARY OF THE INVENTION

The present invention provides a novel wild-type, microbial lipolytic enzyme suitable to be used in the food industry, preferably in the dairy industry, more preferably in the cheese industry. Surprisingly the enzymes herein disclosed have a higher specificity towards short chain fatty acids, in particular C₄-fatty acids and/or C₆-fatty acids, when compared to a known microbial lipolytic enzyme under the same conditions, in particular for the same dosage of enzyme and in the same fat matrix, meaning that these enzyme release or produce or generate more short chain fatty acids than known microbial enzyme(s). This is completely unexpected as, until now, wild-type microbial lipolytic enzymes were known to be specific towards medium or long chain fatty acids and not short chain fatty acids, preferably not specific towards C₄-fatty acids and/or C₆-fatty acids.

This invention relates to a lipase comprising an amino acid sequence
having at least 90% identity with SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11, preferably SEQ ID NO: 1 or SEQ ID NO: 3 or SEQ ID NO: 9 or SEQ ID NO: 11, more preferably SEQ ID NO: 1 or SEQ ID NO: 3 or SEQ ID NO: 11, even more preferably SEQ ID NO: 1, or
having at least 95%, 96%, 97%, 98% or 99% identity SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10 or SEQ ID NO: 11, preferably SEQ ID NO: 1 or SEQ ID NO: 3 or SEQ ID NO: 9 or SEQ ID NO: 11, more preferably SEQ ID NO: 1 or SEQ ID NO: 3 or SEQ ID NO: 11, even more preferably SEQ ID NO: 1 or
wherein the sequence is SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10 or SEQ ID NO: 11, preferably SEQ ID NO: 1 or SEQ ID NO: 3 or SEQ ID NO: 9 or SEQ ID NO: 11, more preferably SEQ ID NO: 1 or SEQ ID NO: 3 or SEQ ID NO: 11, even more preferably SEQ ID NO: 1.

This invention also relates to a lipase comprising an amino acid sequence
having at least 90% identity with SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11, preferably SEQ ID NO: 1 or SEQ ID NO: 3 or SEQ ID NO: 9 or SEQ ID NO: 11, more preferably SEQ ID NO: 1 or SEQ ID NO: 3 or SEQ ID NO: 11, even more preferably SEQ ID NO: 1, or
having at least 95%, 96%, 97%, 98% or 99% identity SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10 or SEQ ID NO: 11, preferably SEQ ID NO: 1 or SEQ ID NO: 3 or SEQ ID NO: 9 or SEQ ID NO: 11, more preferably SEQ ID NO: 1 or SEQ ID NO: 3 or SEQ ID NO: 11, even more preferably SEQ ID NO: 1 or
wherein the sequence is SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10 or SEQ ID NO: 11, preferably SEQ ID NO: 1 or SEQ ID NO: 3 or SEQ ID NO: 9 or SEQ ID NO: 11, more preferably SEQ ID NO: 1 or SEQ ID NO: 3 or SEQ ID NO: 11, even more preferably SEQ ID NO: 1, and
wherein the lipase has a higher specificity towards the release of C₄-fatty acids from a dairy composition comprising milk fat and/or other fat if compared with the release of C₁₀-fatty acids, preferably at a pH below 7, preferably at a pH of 6.6 to 6.8, or at a pH below 6, preferably at a pH between 3.8-5.6, more preferably at a pH between 4.4-5.4, even more preferably at a pH between 4.6-5.2, and/or preferably at a temperature below 40 °C, or below 30 °C, or below 20 °C, preferably below 15 °C, more preferably below 10 °C, even more preferably between 5-8 °C.

This invention also relates to a lipase comprising an amino acid sequence
having at least 90% identity with SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11, preferably SEQ ID NO: 1 or SEQ ID NO: 3 or SEQ ID NO: 9 or SEQ ID NO: 11, more preferably SEQ ID NO: 1 or SEQ ID NO: 3 or SEQ ID NO: 11, even more preferably SEQ ID NO: 1, or 10
having at least 95%, 96%, 97%, 98% or 99% identity SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10 or SEQ ID NO: 11, preferably SEQ ID NO: 1 or SEQ ID NO: 3 or SEQ ID NO: 9 or SEQ ID NO: 11, more preferably SEQ ID NO: 1 or SEQ ID NO: 3 or SEQ ID NO: 11, even more preferably SEQ ID NO: 1 or
wherein the sequence is SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10 or SEQ ID NO: 11, preferably SEQ ID NO: 1 or SEQ ID NO: 3 or SEQ ID NO: 9 or SEQ ID NO: 11, more preferably SEQ ID NO: 1 or SEQ ID NO: 3 or SEQ ID NO: 11, even more preferably SEQ ID NO: 1, and
wherein said lipase has a higher specificity towards the release of C₄-fatty acids from a dairy composition comprising milk fat and/or other fat if compared with the release of C₈-fatty acids, or if compared with the release of C₆-fatty acids, or if compared with the release of C₁₂-fatty acids or if compared with the release of C_{18:2}-fatty acids or if compared with the release of C₁₄-fatty acids or if compared with the release of C_{18:0}-fatty acids or if compared with the release of C_{18:1}-fatty acids or if compared with the release of C₁₆-fatty acids, or if compared with the release of C₈-fatty acids-C_{18:2}-fatty acids, or if compared with the release of C₁₀-fatty acids-C_{18:1}-fatty acids, or if compared with the release of C₁₂-fatty acids-C_{18:0}-fatty acids, or if compared with the release of C₁₄-fatty acids-C_{16:0}-fatty acids, 30
preferably at a pH between 3.8-5.6, more preferably at a pH between 4.4-5.4, even more preferably at a pH between 4.6-5.2 and/or preferably at a temperature below 20 °C, preferably below 15 °C, more preferably below 10 °C, even more preferably between 5-8 °C.

In a preferred embodiment, the lipase herein disclosed—SEQ ID NO: 1 or 2 or 3 or 4 or 5 or 6 or 7 or 8 or 9 or 10 or 11—is an isolated microbial lipase, a recombinant microbial lipase or a synthetic microbial lipase.

Although the present invention relates to a wild-type, non-engineered, microbial lipolytic enzyme the skilled person is aware that one or more alterations may be made to any of the amino acid sequence of the enzymes herein disclosed without interfering with the activity of the said enzyme and preferably without changing the C_{4:0} fatty acid preference of the sequence. Such alteration can be an addition, a substitution or a deletion. For example, a change in the N-terminal or C-terminal of any one of the sequences herein disclose may lead to a different sequence without, however, changing the fatty acid preference of the changed sequence versus the unchanged one. These alterations are herein contemplated as well.

This invention also relates to an isolated DNA sequence or recombinant DNA sequence or synthetic DNA sequence, wherein the isolated DNA sequence or recombinant DNA sequence or synthetic DNA is selected from a sequence having at least 90%, or at least 95%, or at least 96%, or at least 97%, or at least 98% or at least 99%, or 100% sequence identity with SEQ ID NO: 12 or SEQ ID NO: 13 or SEQ ID NO: 14 or SEQ ID NO: 15 or SEQ ID NO: 16 or SEQ ID NO: 17 or SEQ ID NO: 18 or SEQ ID NO: 19 or SEQ ID NO: 20 or SEQ ID NO: 21 or SEQ ID NO: 22 or SEQ ID NO: 23.

In a preferred embodiment, said DNA sequence may further comprise a signal peptide sequence.

This invention further concerns a vector, such as a cloning vector and/or an expression vector, comprising an isolated DNA sequence or recombinant DNA sequence or synthetic DNA sequence encoding the lipase SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11 herein disclosed, or encoding a lipase having at least 90% sequence identity with SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11, or having at least 95%, 96%, 97%, 98% or 99% sequence identity with SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10 or SEQ ID NO: 11. The DNA sequence may be SEQ ID NO: 12 or SEQ ID NO: 13 or SEQ ID NO: 14 or SEQ ID NO: 15 or SEQ ID NO: 16 or SEQ ID NO: 17 or SEQ ID NO: 18 or SEQ ID NO: 19 or SEQ ID NO: 20 or SEQ ID NO: 21 or SEQ ID NO: 22 or SEQ ID NO: 23, or a sequence having at least 90%, or at least 95%, or at least 96%, or at least 97%, or at least 98% or at least 99% sequence identity with SEQ ID NO: 12 or SEQ ID NO: 13 or SEQ ID NO: 14 or SEQ ID NO: 15 or SEQ ID NO: 16 or SEQ ID NO: 17 or SEQ ID NO: 18 or SEQ ID NO: 19 or SEQ ID NO: 20 or SEQ ID NO: 21 or SEQ ID NO: 22 or SEQ ID NO: 23. The sequence is cloned into the vector using standard cloning techniques. The selection of the cloning vector and/or expression vector is dependent on the host cell, as the cloning vector and/or expression vector need to be compatible with the host cell.

The invention also relates to a host cell, preferably a recombinant host cell, comprising SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10, or SEQ ID NO: 11 or SEQ ID NO: 12 or SEQ ID NO: 13 or SEQ ID NO: 14 or SEQ ID NO: 15 or SEQ ID NO: 16 or SEQ ID NO: 17 or SEQ ID NO: 18 or SEQ ID NO: 19 or SEQ ID NO: 20 or SEQ ID NO: 21 or SEQ ID NO: 22 or SEQ ID NO: 23, or comprising a sequence having at least 90% sequence identity with SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11, or having at least 95%, 96%, 97%, 98% or 99% sequence identity with SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10 or SEQ ID NO: 11, or comprising a sequence having at least 90%, or at least 95%, or at least 96%, or at least 97%, or at least 98% or at least 99% sequence identity with SEQ ID NO: 12 or SEQ ID NO: 13 or SEQ ID NO: 14 or SEQ ID NO: 15 or SEQ ID NO: 16 or SEQ ID NO: 17 or SEQ ID NO: 18 or SEQ ID NO: 19 or SEQ ID NO: 20 or SEQ ID NO: 21 or SEQ ID NO: 22 or SEQ ID NO: 23.

The invention also concerns a method of growing, transforming or transfecting said vector in a suitable host cell and in a condition leading to expression of the polypeptide or to the over-expression of the polypeptide.

A suitable host cell used in this invention may be any host cell in which a recombinant DNA can be replicated and a gene encoding an enzyme can be expressed. Preferably, the host cell may be a mammalian cell, an insect cell, a microbial cell, e.g. a bacterial cell or a fungal cell wherein a yeast cell is included.

Examples of suitable yeasts may be Saccharomyces spp., Schizosaccharomyces spp., Candida spp., Pichia spp., Kluyveromyces spp., such as Saccharomyces cerevisiae; Candida cylindracea, or Pichia pastoris.

Examples of suitable fungal cells, including filamentous fungal cells, may be selected from Fusarium spp., Aspergillus spp., Trichoderma spp., such as Fusarium oxysporium, Aspergillus oryzae or Aspergillus niger.

Examples of suitable bacteria are Gram-negative such as Escherichia coli or Gram-positive bacteria selected from Bacillus spp., Streptomyces spp., Corynebacterium spp., such as Bacillus subtilis, Bacillus licheniformis, Bacillus lentus, Bacillus brevis, Bacillus stearothermophilus, Bacillus alkalophilus, Bacillus amyloliquefaciens, Bacillus coagulans, Bacillus circulans, Bacillus lautus, Bacillus megaterium, Bacillus thuringiensis; Streptomyces lividans or Streptomyces murinus. The transformation of bacteria may, for instance, be effected by using competent cells in a manner known in the art or by any other way known in the art.

In a preferred embodiment, the host cell is selected from Pichia pastoris, Saccharomyces spp., Saccharomyces cerevisiae, Schizosaccharomyces spp., Candida spp., Candida cylindracea, Kluyveromyces spp., Fusarium spp., Fusarium oxysporium, Aspergillus spp., Aspergillus oryzae or Aspergillus niger, Trichoderma spp., Escherichia coli or Bacillus spp., Bacillus subtilis, Bacillus licheniformis, Bacillus lentus, Bacillus brevis, Bacillus stearothermophilus, Bacillus alkalophilus, Bacillus amyloliquefaciens, Bacillus coagulans, Bacillus circulans, Bacillus lautus, Bacillus megaterium, Bacillus thuringiensis, Streptomyces spp., Streptomyces lividans or Streptomyces murinus, Corynebacterium spp., preferably Pichia pastoris, Aspergillus oryzae or Aspergillus niger, or Bacillus subtilis, more preferably Pichia pastoris or Bacillus subtilis.

The invention also relates to a lipase consisting of an amino acid sequence having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11, preferably having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 9 or SEQ ID NO: 11; wherein the lipase is expressed in Pichia pastoris and has a higher specificity towards the release of C₄-fatty acids from a dairy composition comprising milk fat and/or other fat if compared with the release of C₁₀-fatty acids, or if compared with the release of C₈-fatty acids, or if compared with the release of C₆-fatty acids, or if compared with the release of C₁₂-fatty acids or if compared with the release of C_{18:2}-fatty acids or if compared with the release of C₁₄-fatty acids or if compared with the release of C_{18:0}-fatty acids or if compared with the release of C_{18:1}-fatty acids or if compared with the release of C₁₆-fatty acids, or if compared with the release of C₈-fatty acids-C_{18:2}-fatty acids, or if compared with the release of C₁₀-fatty acids-C_{18:1}-fatty acids, or if compared with the release of C₁₂-fatty acids-C_{18:0}-fatty acids, or if compared with the release of C₁₄-fatty acids-C_{16:0}-fatty acids or if compared with the release of C₁₆-C₁₈-fatty acids.

The present invention also concerns a method for preparing a food product
comprising a step of using or adding a lipase comprising an amino acid sequence having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11, preferably having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 9 or SEQ ID NO: 11, preferably, wherein the food product has a higher content or amount or concentration of short chain fatty acids, such as C₄- and/or C₆-fatty acids, than if compared with the content or amount or concentration of medium to long chain fatty acids; or
comprising a step of using or adding a lipase encoded by a DNA sequence selected from a sequence having at least 90%, or at least 95%, or at least 96%, or at least 97%, or at least 98% or at least 99%, or 100% sequence identity with SEQ ID NO: 12 or SEQ ID NO: 13 or SEQ ID NO: 14 or SEQ ID NO: 15 or SEQ ID NO: 16 or SEQ ID NO: 17 or SEQ ID NO: 18 or SEQ ID NO: 19 or SEQ ID NO: 20 or SEQ ID NO: 21 or SEQ ID NO: 22 or SEQ ID NO: 23, preferably having at least 90%, or at least 95%, or at least 96%, or at least 97%, or at least 98% or at least 99%, or 100% sequence identity with SEQ ID NO: 12 or SEQ ID NO: 13 or SEQ ID NO: 14 or SEQ ID NO: 20 or SEQ ID NO: 22 or SEQ ID NO: 23, preferably wherein the food product has a higher content or amount or concentration of short chain fatty acids, such as C₄- and/or C₆-fatty acids, than if compared with the content or amount or concentration of medium to long chain fatty acids.

In a preferred embodiment, the food product may be a dairy product, as a cheese product, a processed cheese, a cheese-like product, or an enzyme-modified cheese, or a butter, a yogurt, a cream, a seasoning.

In a preferred embodiment, the dairy product is a cheese product, preferably wherein the cheese product is Pecorino, Provolone, Parmesan, Grana Padano, Parmigiano Reggiano, Romano, Chester, Danbo, Manchego, Saint Paulin, Cheddar, Monterey, Colby, Edam, Gouda, Muenster, Swiss type, Gruyere, Emmental; curd-cheeses such as Feta cheese; pasta filata cheeses such as Mozzarella, and Queso fresco cheese; fresh cheese such as Ricotta, Cream cheese, Neufchatel or Cottage cheese; cream cheese, white mold cheese such as Brie and Camembert cheese, blue mold cheese such as Gorgonzola and Danish blue cheese; and processed cheese, preferably Feta cheese or Provolone cheese.

In another embodiment, the food product may be a non-dairy product, preferably a non-dairy cheese or vegan cheese, more preferably wherein the lipase has a higher specificity towards the release of C₄-fatty acids from a composition comprising fat such as vegetable fat or from a water-in-oil emulsion if compared with the release of C₁₀-fatty acids or if compared with the release of C₈-fatty acids, or if compared with the release of C₆-fatty acids, or if compared with the release of C₁₂-fatty acids or if compared with the release of C_{18:2}-fatty acids or if compared with the release of C₁₄-fatty acids or if compared with the release of C_{18:0}-fatty acids or if compared with the release of C_{18:1}-fatty acids or if compared with the release of C₁₆-fatty acids, or if compared with the release of C₈-fatty acids-C_{18:2}-fatty acids, or if compared with the release of C₁₀-fatty acids-C_{18:1}-fatty acids, or if compared with the release of C₁₂-fatty acids-C_{18:0}-fatty acids, or if compared with the release of C₁₄-fatty acids-C_{16:0}-fatty acids or if compared with the release of C₁₆-C₁₈-fatty acids.

In a preferred embodiment, cheese flavor or cheese intensity can be increased and/or improved by applying a lipase of the present invention. The increase or improvement of cheese flavor or cheese intensity can apply to cheese itself or to processed cheese, to cheese-like product or to enzyme-modified cheese or even to non-dairy cheese such as vegan cheese.

In a preferred embodiment, the lipases herein disclosed may be added, for example, to an intermediate product during the process of producing a food product or to a raw material such as milk, in particular milk obtained from a cow, sheep, goat, among others.

The invention also concerns the use of a lipase comprising an amino acid sequence having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11, preferably having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 9 or SEQ ID NO: 11, for producing a food product or as an agent for producing a food product, preferably wherein the food product has a higher content or amount or concentration of short chain fatty acids, such as C₄- and/or C₆-fatty acids, than if compared with the content or amount or concentration of medium to long chain fatty acids, and wherein the food product is as a dairy product or a cheese product, a processed cheese, a cheese-like product, or an enzyme-modified cheese, or a butter, a yogurt, a cream, a seasoning.

In a preferred embodiment, the use of a lipase is for producing a food product, in particular a dairy product or a cheese such Pecorino, Provolone, Parmesan, Grana Padano, Parmigiano Reggiano, Romano, Chester, Danbo, Manchego, Saint Paulin, Cheddar, Monterey, Colby, Edam, Gouda, Muenster, Swiss type, Gruyere, Emmental; curd-cheeses such as Feta cheese; pasta filata cheeses such as Mozzarella, and Queso fresco cheese; fresh cheese such as Ricotta, Cream cheese, Neufchatel or Cottage cheese; cream cheese, white mold cheese such as Brie and Camembert cheese, blue mold cheese such as Gorgonzola and Danish blue cheese, preferably Feta cheese or Provolone cheese; or processed cheese.

The invention also relates to the use of a lipase for producing or releasing more C₄-fatty acids from a dairy composition comprising milk fat and/or other fat, than for producing or releasing more C₆-fatty acids, C₈-fatty acids, C₁₀-fatty acids, C₁₂-fatty acids, C₁₄-fatty acids, C₁₆-fatty acids, C_{18:0}-fatty acids, C_{18:1}-fatty acids, or C_{18:2}-fatty acids, or C_{18:3}-fatty acids, wherein the lipase comprises an amino acid sequence having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11, preferably having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 9 or SEQ ID NO: 11.

The invention also concerns the use of a lipase for producing or releasing more C₄-fatty acids and/or C₆-fatty acids in a food product than for producing or releasing, C₈-fatty acids, C₁₀-fatty acids, C₁₂-fatty acids, C₁₄-fatty acids, C₁₆-fatty acids, C_{18:0}-fatty acids, C_{18:1}-fatty acids, or C_{18:2}-fatty acids, or C_{18:3}-fatty acids in the food product, wherein the lipase comprises an amino acid sequence having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11, preferably having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 9 or SEQ ID NO: 11,
preferably wherein the food product is a dairy product selected from a cheese, or a processed cheese, or enzyme-modified cheese, or a cheese-like product, or a butter, or a yogurt, or a cream, or a seasoning, or
preferably wherein the food product is a non-dairy product such as a non-dairy cheese or vegan cheese.

In an embodiment, the food product is a dairy product selected from a cheese and wherein the cheese is Feta cheese, or Provolone cheese, or Pecorino cheese, or Parmesan cheese, or Grana Padano cheese, or Parmigiano Reggiano cheese, or Romano cheese, or Chester cheese, or Danbo cheese, or Manchego cheese, or Saint Paulin cheese, or Cheddar cheese, or Monterey cheese, or Colby cheese, or Edam cheese, or Gouda cheese, or Muenster cheese, or Swiss type cheese, or Gruyere cheese, or Emmental cheese; or pasta filata cheeses such as Mozzarella, and Queso fresco cheese; or fresh cheese such as Ricotta, Cream cheese, Neufchatel or Cottage cheese; or cream cheese, or white mold cheese such as Brie and Camembert cheese, or blue mold cheese such as Gorgonzola and Danish blue cheese, preferably Feta cheese or Provolone cheese.

Furthermore, the invention concerns a food product obtainable by using a lipase SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11, preferably wherein the food product is a dairy product such as a cheese product, a processed cheese, a cheese-like product, or a butter, a yogurt, a cream, a seasoning, more preferably wherein the dairy product is a cheese, such Pecorino, Provolone, Parmesan, Grana Padano, Parmigiano Reggiano, Romano, Chester, Danbo, Manchego, Saint Paulin, Cheddar, Monterey, Colby, Edam, Gouda, Muenster, Swiss type, Gruyere, Emmental; curd-cheeses such as Feta cheese; pasta filata cheeses such as Mozzarella, and Queso fresco cheese; fresh cheese such as Ricotta, Cream cheese, Neufchatel or Cottage cheese; cream cheese, white mold cheese such as Brie and Camembert cheese, blue mold cheese such as Gorgonzola and Danish blue cheese; and processed cheese, preferably Feta cheese or Provolone cheese.

The invention is related as well with a food product comprising a lipase, wherein the lipase comprises an amino acid sequence having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11, preferably having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 9 or SEQ ID NO: 11, and
wherein the food product comprises more C₄-fatty acids than C₁₀-fatty acids or than any other fatty acid selected from C₆-fatty acids or C₈-fatty acids or C₁₂-fatty acids or C₁₄-fatty acids or C₁₆-fatty acids or C_{18:1}-fatty acids or C_{18:2}-fatty acids or C_{18:3}-fatty acids and/or
wherein the food product comprises more C₆-fatty acids than C₁₀-fatty acids or than any other fatty acid selected from C₈-fatty acids or C₁₂-fatty acids or C₁₄-fatty acids or C₁₆-fatty acids or C_{18:1}-fatty acids or C_{18:2}-fatty acids or C_{18:3}-fatty acids.

In an embodiment, the food product is a dairy product selected from a cheese, a processed cheese, a cheese-like product, an enzyme-modified cheese, or a butter, a yogurt, a cream, a seasoning, preferably wherein the cheese is Feta cheese, or Provolone cheese, or Pecorino cheese, or Parmesan cheese, or Grana Padano cheese, or Parmigiano Reggiano cheese, or Romano cheese, or Chester cheese, or Danbo cheese, or Manchego cheese, or Saint Paulin cheese, or Cheddar cheese, or Monterey cheese, or Colby cheese, or Edam cheese, or Gouda cheese, or Muenster cheese, or Swiss type cheese, or Gruyere cheese, or Emmental cheese; or pasta filata cheeses such as Mozzarella, and Queso fresco cheese; or fresh cheese such as Ricotta, Cream cheese, Neufchatel or Cottage cheese; or cream cheese, or white mold cheese such as Brie and Camembert cheese, or blue mold cheese such as Gorgonzola and Danish blue cheese, more preferably Feta cheese or Provolone cheese.

In another embodiment, the food product is a non-dairy product such as a non-dairy cheese or vegan cheese.

The invention also relates to a composition comprising a lipase according to SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11, and/or a DNA sequence according SEQ ID NO: 12 or SEQ ID NO: 13 or SEQ ID NO: 14 or SEQ ID NO: 15 or SEQ ID NO: 16 or SEQ ID NO: 17 or SEQ ID NO: 18 or SEQ ID NO: 19 or SEQ ID NO: 20 or SEQ ID NO: 21 or SEQ ID NO: 22 or SEQ ID NO: 23, and/or a vector comprising any of the sequences SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11 or SEQ ID NO: 12 or SEQ ID NO: 13 or SEQ ID NO: 14 or SEQ ID NO: 15 or SEQ ID NO: 16 or SEQ ID NO: 17 or SEQ ID NO: 18 or SEQ ID NO: 19 or SEQ ID NO: 20 or SEQ ID NO: 21 or SEQ ID NO: 22 or SEQ ID NO: 23 and/or a host cell comprising any of the sequences SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11 or SEQ ID NO: 12 or SEQ ID NO: 13 or SEQ ID NO: 14 or SEQ ID NO: 15 or SEQ ID NO: 16 or SEQ ID NO: 17 or SEQ ID NO: 18 or SEQ ID NO: 19 or SEQ ID NO: 20 or SEQ ID NO: 21 or SEQ ID NO: 22 or SEQ ID NO: 23, preferably wherein the composition is for preparing a food product, such as a dairy product or cheese.

### DEFINITIONS

In the context of the present invention the terms “short chain fatty acid”, “medium chain fatty acid”, “long chain fatty acid”, “FFA”, “lipase”, “lipolytic enzyme”, “microbial lipase”, “microbial lipolytic enzyme”, “lipase with higher specificity towards the release of short-chain fatty acids”, “lipase with higher specificity towards the release of C₄-fatty acids”, “lipase with higher specificity towards the release of C₆-fatty acids”, lipase with higher specificity towards the release of medium and/or long-chain fatty acids”, “lipase activity”, “lipase dosage”, “wild type”, “synthetic sequence”, “isolated sequence”, “recombinant sequence”, “mature protein sequence”, “expression vector”, “sequence identity”, “food product”, “dairy product”, “cheese”, “cheese product”, “processed cheese”, “enzyme-modified cheese”, “cheese-like product”, “improvement factor (IF)”, “control microbial lipase”, “signal peptide”, “propeptide” have the meaning explained below.

The term “short chain fatty acid” means a fatty acid comprising 4-6 carbons. Examples of short chain fatty acids are: butyric acid (butanoic acid; C₄), valeric acid (pentanoic acid; C₅) or caproic acid (hexanoic acid; C₆).

The term “medium chain fatty acid” means a fatty acid comprising 8-12 carbons. Examples of medium chain fatty acids are: caprylic acid (octanoic acid; C₈), pelargonic acid (nonanoic acid; C₉), capric acid (decanoic acid; C₁₀), undecylic acid (undecanoic acid; C₁₁) or lauric acid (dodecanoic acid; C₁₂).

The term “long chain fatty acid” means a fatty acid comprising 14-18 or more carbons. Examples of long chain fatty acids are: myristic acid (tetradecanoic acid; C₁₄), pentadecanoic acid (C₁₅), palmitic acid (hexadecanoic acid; C₁₆), margaric acid (heptadecanoic acid; C₁₇), stearic acid (octadecanoic acid; C₁₈).

The term “FFA” stands for free fatty acid.

The term “lipase” and “lipolytic enzyme” are used interchangeably.

The term “microbial lipase” and “microbial lipolytic enzyme” are used interchangeably, and as used herein means a lipase or a lipolytic enzyme expressed by a naturally occurring microorganism found in nature. Furthermore, the microorganism may be a filamentous fungus and/or a non-filamentous fungus. Thus, the lipase or the microbial lipase or the microbial lipolytic enzyme may be a filamentous fungus lipase and/or non-filamentous fungus lipase. The term “lipase with higher specificity towards the release of short-chain fatty acids”, from a dairy composition comprising milk fat and/or other fat, means that said lipase preferably hydrolyzes ester bonds of lipids of triglycerides generating more C₄-fatty acids and/or more C₆-fatty acids rather than generating medium and/or long chain fatty acids.

The term “lipase with higher specificity towards the release of C₄-fatty acids”, from a dairy composition comprising milk fat and/or other fat, means that said lipase preferably hydrolyzes ester bonds of lipids of triglycerides generating more C₄-fatty acids rather than generating other kinds of fatty acids, such as medium and/or long chain fatty acids. Preferably, the term “lipase with higher specificity towards the release of C₄-fatty acids”, from a dairy composition comprising milk fat and/or other fat, if compared with the release of C₁₀-fatty acids means that said lipase preferably hydrolyses ester bonds of lipids of triglycerides generating more C₄-fatty acids rather than generating C₁₀-fatty acids. More preferably, the term “lipase with higher specificity towards the release of C₄-fatty acids”, from a dairy composition comprising milk fat and/or other fat, if compared with the release of C₈-fatty acids or C₆-fatty acids or C₁₂-fatty acids or C_{18:2}-fatty acids or C₁₄-fatty acids or C_{18:0}-fatty acids or C_{18:1}-fatty acids or C₁₆-fatty acids means that said lipase preferably hydrolyses ester bonds of lipids of triglycerides generating more C₄-fatty acids rather than generating C₈-fatty acids or C₆-fatty acids or C₁₂-fatty acids or C_{18:2}-fatty acids or C₁₄-fatty acids or C_{18:0}-fatty acids or C_{18:1}-fatty acids or C₁₆-fatty acids, respectively. In particular, the molar fraction of C₄-fatty acids is higher than the molar fraction of medium and/or long chain fatty acids, for the same tested conditions, preferably the molar fraction of C₄-fatty acids is higher than the molar fraction of other fatty acids, such as C₆-fatty acids or C₈-fatty acids or C₁₀-fatty acids or C₁₂-fatty acids or C₁₄-fatty acids or C₁₆-fatty acids or C_{18:0}-fatty acids or C_{18:1}-fatty acids or C_{18:2}-fatty acids.

The term “lipase with higher specificity towards the release of C₄-fatty acids”, from a dairy composition comprising milk fat and/or other fat, also means that said lipase preferably hydrolyzes ester bonds of lipids of triglycerides generating more C₄-fatty acids than a control microbial lipase. In particular, the molar fraction of C₄-fatty acids of the tested lipase is higher than the molar fraction of C₄-fatty acids of the control microbial lipase.

The term “lipase with higher specificity towards the release of C₆-fatty acids”, from a dairy composition comprising milk fat and/or other fat, means that said lipase preferably hydrolyzes ester bonds of lipids of triglycerides generating more C₆-fatty acids rather than generating other kinds of fatty acids, such as medium and/or long chain fatty acids. Preferably, the term “lipase with higher specificity towards the release of C₆-fatty acids”, from a dairy composition comprising milk fat and/or other fat, if compared with the release of C₁₀-fatty acids means that said lipase preferably hydrolyses ester bonds of lipids of triglycerides generating more C₆-fatty acids rather than generating C₁₀-fatty acids. More preferably, the term “lipase with higher specificity towards the release of C₆-fatty acids”, from a dairy composition comprising milk fat and/or other fat, if compared with the release of C₈-fatty acids or C₁₂-fatty acids or C_{18:2}-fatty acids or C₁₄-fatty acids or C_{18:0}-fatty acids or C_{18:1}-fatty acids or C₁₆-fatty acids means that said lipase preferably hydrolyses ester bonds of lipids of triglycerides generating more C₆-fatty acids rather than generating C₈-fatty acids or C₁₂-fatty acids or C_{18:2}-fatty acids or C₁₄-fatty acids or C_{18:0}-fatty acids or C_{18:1}-fatty acids or C₁₆-fatty acids, respectively. In particular, the molar fraction of C₆-fatty acids is higher than the molar fraction of medium and/or long chain fatty acids, for the same tested conditions, preferably the molar fraction of C₆-fatty acids is higher than the molar fraction of other fatty acids, such as C₈-fatty acids or C₁₀-fatty acids or C₁₂-fatty acids or C₁₄-fatty acids or C₁₆-fatty acids or C_{18:0}-fatty acids or C_{18:1}-fatty acids or C_{18:2}-fatty acids.

The term “lipase with higher specificity towards the release of C₆-fatty acids”, from a dairy composition comprising milk fat and/or other fat, also means that said lipase preferably hydrolyzes ester bonds of lipids of triglycerides generating more C₆-fatty acids than a control microbial lipase. In particular, the molar fraction of C₆-fatty acids of the tested lipase is higher than the molar fraction of C₄-fatty acids of the control microbial lipase.

The term “lipase with higher specificity towards the release of medium and/or long-chain fatty acids”, from a dairy composition comprising milk fat and/or other fat, means that said lipase preferably hydrolyzes ester bonds of lipids of triglycerides generating C₈- to C₁₈-fatty acids rather than generating C₄-fatty acids and/or C₆-fatty acids. In particular, the molar fraction of C₈- to C₁₈-fatty acids of the tested lipase is higher than the molar fraction of C₈- to C₁₈-fatty of the control microbial lipase.

In the context of the present inventions the following terms are interchangeable “C₄-fatty acids” and “C_{4:0}-fatty acids”; “C₆-fatty acids” and “C_{6:0}-fatty acids”; “C₈-fatty acids” and “C_{8:0}-fatty acids”; “C₁₀-fatty acids” and “C_{10:0}-fatty acids”; “C₁₂-fatty acids” and “C_{12:0}-fatty acids”; “C₁₄-fatty acids” and “C_{14:0}-fatty acids”. The term “C₁₆-fatty acids” may include C_{16:0}-fatty acids and C_{16:1}-fatty acids”. The term “C₁₈-fatty acids” may include C_{18:0}-fatty, C_{18:1}-fatty acids and C_{18:3}-fatty.

The term “lipase activity” and “lipase dosage” are used interchangeably. The “lipase activity” may be determined, for dosing in cheese and cream, for example by the International Dairy Federation (IDF) method for both the commercial lipase and the enzymes herein disclosed (SEQ ID NO 1-11). The IDF method, mainly, corresponds to the International Standard ISO 13082:2011, 1^{st} edition of 2011 Nov. 15, IDF 218:2011 (also labelled as ISO 13082|218). However, in the present invention, the term “LFU/L of milk” is used. The term “LFU/L of milk” relates to the concentration or amount of enzyme or dosage of enzyme per liter of milk used to produce a dairy product, such as cheese. In the context of the present invention “LFU/L of milk” means lipase forestomach units per liter of milk. One LFU is defined as the amount of lipase activity that releases butyric acid at a rate of 1.25 μmol/min, under the tested conditions. Further, the lipase activity may also be determined as explained in the Examples below. Finally, there are other methods of determining lipase activity which has been disclosed in prior art documents (such as in EP 3 081 644, EP 2 254 996 or EP 1 776 455). However, what is relevant is that within the same example or within comparative examples, the lipase activity is determined in the same way for all lipases under analysis, including the control lipase.

The term “wild type” lipase means, in the context of the present invention, a lipase whose sequence has not been mutated, i.e. does not contain amino acid deletions, additions or substitutions, when compared with a mature protein sequence (after co- and/or post-translational cleavage events) endogenously produced. Wild type lipases of the present invention may be encoded by codon optimized polynucleotides for heterologous expression and may also comprise a non-endogenous signal- and/or propeptide selected for expression in a given host. Furthermore, in the present invention, a lipase may also be synthetically obtained or genetically modified, using methods well known in the art, as to reproduce the wild-type lipase.

The term “synthetic sequence” or a variation thereof means a sequence that has been prepared by in vitro chemical or enzymatic synthesis. It includes, but is not limited to, sequences made with optimal codon usage for host organisms. The term applies to a synthetic microbial lipase or to a synthetic DNA sequence.

The term “isolated sequence” or a variation thereof means a sequence which is at least substantially free from at least one other component with which the sequence is naturally associated in nature and as found in nature. In alternative, an “isolated sequence” can also mean a sequence removed from its native environment. For example, sequences which have been recombinantly produced in a host organism are considered isolated for the purpose of this invention as are native or recombinant sequences which have been substantially purified by any suitable technique as such, for example, a purification method. Purification methods are well known in the art. The term applies to an isolated microbial lipase or to an isolated DNA sequence.

The term “recombinant sequence” or a variation thereof means a sequence that has been prepared using recombinant DNA techniques, which are within the capabilities of a person skilled in the art and are explained in literature, for example, J. Sambrook, E. F. Fritsch, and T. Maniatis, 1989, Molecular Cloning: A Laboratory manual, 2nd edition, books 1-3, Cold Spring Harbor Laboratory Press. The term applies to a recombinant microbial lipase or to a recombinant DNA sequence.

The term “mature protein sequence” used herein means a sequence having lipolytic activity that is in its final form following translation and any co- and/or post-translational cleavage events or modifications, such as N-terminal processing, C-terminal truncation, glycosylation, phosphorylation, among other events or modifications.

The term “expression vector” means a construct capable of in vivo or in vitro expression. The expression vector may be incorporated, in particular stably incorporated, into the genome of a suitable host organism.

The term “sequence identity” describes a quantitative measure of similarity between two amino acid sequences or two nucleotide sequences. For purposes of the present invention, the degree of sequence identity between two amino acid sequences is based on aligning both sequences with the blastp suite provided by the National center for Biotechnology Information (NCBI) on https://blast.ncbi.nlm.nih.gov applying standard parameter settings (Matrix: BLOSUM62, Gap Costs: Existence: 11 Extension: 1, Conditional compositional score matrix adjustment) and subsequent quantification of identical amino acid pairs in identical positions over the aligned amino acid sequences.

The term “food product” refers to a kind of milk-based product intended to be used as food, including, but not limited to, cheese, milk, skimmed milk, acidified milk, butter milk, condensed milk, spread, margarine, yoghurt, ice cream, milk powder, butter, dulce de leche, among others.

The term “dairy product” is intended to include any food product made using milk or milk-based product, including, but not limited to, cheese, milk, skimmed milk, acidified milk, butter milk, condensed milk, spread, margarine, yoghurt, ice cream, milk powder, butter, dulce de leche, among others.

The term “cheese” or “cheese product” are used interchangeably. The term “cheese” is understood to encompass any cheese, including, but not limited to, hard cheeses such as Pecorino, Provolone, Parmesan, Grana Padano, Parmigiano Reggiano, Romano, Chester, Danbo, Manchego, Saint Paulin, Cheddar, Monterey, Colby, Edam, Gouda, Muenster, Swiss type, Gruyere, Emmental; curd-cheeses such as Feta cheese; pasta filata cheeses such as Mozzarella, and Queso fresco cheese; fresh cheese such as Ricotta, Cream cheese, Neufchatel or Cottage cheese; cream cheese, white mold cheese such as Brie and Camembert cheese, blue mold cheese such as Gorgonzola and Danish blue cheese; and processed cheese, enzyme-modified cheese (EMC) or cheese-like product.

The term “processed cheese” is preferably manufactured from cheese or cheese analogues by cooking and emulsifying the cheese, such as with emulsifying salts (e.g. phosphates and citrate). The process may further include the addition of spices/condiments.

The term “enzyme-modified cheese” or “EMC” is understood as cheese curd which has been treated with enzymes to produce a concentrated cheese flavor ingredient which may have approximately 15-30 times the flavor intensity of natural cheese. EMCs are available as pastes or dried to form powders and are used to give a cheese flavor note to products such as processed cheese or analogue cheese, cheese powders, soups, sauces, dips, crackers, salad dressings and in coatings for snack foods.

The term “cheese-like product” is understood as cheese-like products which contain fat, such as e.g. milk fat (e.g. cream or butter) or vegetable oil, as a part of the composition, and which further contain, as part of the composition, one or more non-milk constituents, such as e.g. a vegetable constituent (e.g. vegetable protein or vegetable oil). In the context of the present invention, a “cheese-like product” includes a non-dairy cheese, also known as vegan cheese, wherein the fat used to make the non-dairy cheese is vegetable oil or an emulsion such as water-in-oil emulsion, instead of milk fat.

The term “improvement factor (IF)” is defined as the ratio obtained when dividing the enzymatic specificity for the release of short chain fatty acids of a tested lipase by the enzymatic specificity for the release of short chain fatty acids of a control lipase, preferably a control microbial lipase or is defined as the ratio obtained when dividing the enzymatic specificity for the release of medium chain fatty acids of a tested lipase by the enzymatic specificity for the release of medium chain fatty acids of a control lipase, preferably a control microbial lipase. If the ratio is 1, then the tested lipase has an identical preference, or specificity, for short chain fatty acids as control sequence. If the ratio is lower than one, then the tested lipase has less preference, or specificity, for short chain fatty acids than the control lipase, preferably a control microbial lipase. If the ratio is higher than one, then the tested lipase has higher preference, or specificity, for short chain fatty acids than the control. The enzymatic specificity for short chain fatty acids is obtained by dividing the enzymatic activity for short chain fatty acids C₄ or C₆ by the enzymatic activity for long chain fatty acids C₁₆-C₁₈. The improvement factor (IF) may also be determined by the ratio obtained when dividing the enzymatic specificity for the release of long chain fatty acids of a tested lipase by the enzymatic specificity for the release of long chain fatty acids of a control lipase, preferably a control microbial lipase. If the ratio is 1, then the tested lipase has an identical preference, or specificity, for longer chain fatty acids as the control lipase, preferably a control microbial lipase. If the ratio is lower than one, then the tested lipase has less preference, or specificity, for long chain fatty acids than the control. If the ratio is higher than one, then the tested lipase has higher preference, or specificity, for long chain fatty acids than the control. The enzymatic specificity for long chain fatty acids is obtained by dividing the enzymatic activity for long chain fatty acids C₁₆-C₁₈ by the enzymatic activity for short chain fatty acids C₄ or C₆.

The term “control lipase” or “control microbial lipase” or “control lipolytic enzyme” or “control microbial lipolytic enzyme” is a commercial microbial lipase from Rhizomucur miehei (Palatase®, Palatase® 20000 L, available from Novozymes A/S), and herein labelled as commercial microbial lipase or commercial microbial lipase A. Alternatively, other microbial lipases from Rhizomucur miehei can be used, for example SpiceIT® MR available from Chr. Hansen.

The term “signal peptide” relates to an amino acid sequence fused to the N-terminus of the polypeptide chain of the enzyme that is recognized by the secretory pathway of the enzyme producing microbial organism to translocate the synthesized protein into the cellular membrane or secrete it out of the cell into the fermentation medium.

The term “propeptide” relates to a polypeptide chain fused to the N-terminus of the major peptide chain of an enzyme that may support folding of the synthesized protein and/or contribute to its stability. The propeptide is cleaved during maturation or activation of the enzyme. A propeptide acts as an N-terminal extension of protein, distinct from the signal sequence which is necessary for the transport of the protein into or through the membrane, or for its secretion into the extracellular medium. The presence of a propeptide sequence may lead to a stabilization of the expression of lipases and/or may prevent inactivation in vivo of the lipase.

Amino acids are herein indicated by their one letter code or by their three letter code, both of which are well known to a person skilled in the art.

In the context of the present invention, SEQ ID NO: 1-11 stands for SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 and SEQ ID NO: 11. Further, in the context of this invention, SEQ ID NO: 12-23 stands for SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22 and SEQ ID NO: 23.

### DETAILED DESCRIPTION

The present invention relates to a non-animal, wild-type derived from a microorganism lipase fulfilling the vegetarian and/or kosher requirements while simultaneously presenting a specificity and preference for the cleavage of short chain fatty acids, preferably presenting a higher specificity towards C₄-fatty acids and/or C₆-fatty acids rather other kinds of fatty acids or presenting a higher specificity towards C₄-fatty acids and/or C₆-fatty acids than a commercial microbial lipase.

The purpose of this invention is to provide a microbial wild-type lipase with a higher specificity towards the release of C₄-fatty acids and/or C₆-fatty acids, from a dairy composition comprising milk fat and/or other fat, if compared with the release of medium and/or long chain fatty acids, preferably if compared with the release of long-chain fatty acids, or a higher specificity towards the release of C₄-fatty acids and/or C₆-fatty acids, from a dairy composition comprising milk fat and/or other fat, if compared to the release of C₄-fatty acids and/or C₆-fatty acids of a commercial microbial lipase, without the need to generate any mutation (addition, deletion and/or substitution) in the lipase sequence. Thus, this invention relates to an improved (or alternative) microbial wild-type lipase with specificity towards short-chain fatty acids versus the prior microbial lipases. Therefore, the lipases herein disclosed may be obtained from microorganisms that produce the lipase naturally. In this case, said lipase is a wild-type lipase.

The microbial lipases wherein disclosed (SEQ ID NO: 1-11) may be used to replace the microbial lipases currently available on the market.

### EXAMPLES

### Example 1—Cloning and Expression

A nucleotide sequence encoding for SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11 was integrated into a targeted locus of an expression host such as Pichia pastoris. Codon-optimized genes encoding for SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11 may or not be used. The nucleotide sequence encoding for SEQ ID NO: 1 is, for example, SEQ ID NO: 12 or SEQ ID NO: 23. The nucleotide sequence encoding for SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11 can be, for example, SEQ ID NO: 13 or SEQ ID NO: 14 or SEQ ID NO: 15 or SEQ ID NO: 16 or SEQ ID NO: 17 or SEQ ID NO: 18 or SEQ ID NO: 19 or SEQ ID NO: 20, or SEQ ID NO: 21 or SEQ ID NO: 22, respectively.

In an embodiment Pichia pastoris may be replaced by Saccharomyces spp., Saccharomyces cerevisiae, Schizosaccharomyces spp., Candida spp., Candida cylindracea, Kluyveromyces spp., Fusarium spp., Fusarium oxysporium, Aspergillus spp., Aspergillus oryzae or Aspergillus niger, Trichoderma spp., Escherichia coli or Bacillus spp., Bacillus subtilis, Bacillus licheniformis, Bacillus lentus, Bacillus brevis, Bacillus stearothermophilus, Bacillus alkalophilus, Bacillus amyloliquefaciens, Bacillus coagulans, Bacillus circulans, Bacillus lautus, Bacillus megaterium, Bacillus thuringiensis, Streptomyces spp., Streptomyces lividans or Streptomyces murinus, Corynebacterium spp., preferably Aspergillus oryzae, Aspergillus niger, or Bacillus subtilis.

A nucleotide sequence encoding for SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11 may also be cloned into a conventional cloning and expression vector. Codon-optimized genes encoding for SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11 may or not be used. The nucleotide sequence encoding for SEQ ID NO: 1 may be, for example, SEQ ID NO: 12 or SEQ ID NO: 23. The nucleotide sequence encoding for SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11 can be, for example, SEQ ID NO: 13 or SEQ ID NO: 14 or SEQ ID NO: 15 or SEQ ID NO: 16 or SEQ ID NO: 17 or SEQ ID NO: 18 or SEQ ID NO: 19 or SEQ ID NO: 20, or SEQ ID NO: 21 or SEQ ID NO: 22, respectively.

The recombinant cloning and expression vector may be further introduced in a host cell, such as a recombinant host cell, using standard transformation or transfection protocols known to the person skilled in the art and explained in literature (for example, in J. Sambrook, E. F. Fritsch, and T. Maniatis, 1989, Molecular Cloning: A Laboratory manual, 2^{nd} edition, books 1-3, Cold Spring Harbor Laboratory Press).

The microbial lipases (SEQ ID NO: 1-11) herein disclosed were independently expressed as follows. Pichia pastoris was used as a host for lipase expression. Pichia pastoris was grown on YPD agar plates (Invitrogen) under selective conditions (100 μg/ml Zeocin) at 30 °C for 16 h. Seed fermentation was performed by inoculating 3 mL YPD liquid medium (Invitrogen) including 100 μg/mL Zeocin with a single colony from the respective YPD agar plates, followed by incubation at 30 °C and 250 rpm agitation for 16 h. Main fermentation was performed in 4 mL total volume by diluting seed fermentations with BMGY medium (Invitrogen) including 100 μg/mL Zeocin to an OD₆₀₀ of 0.2, followed by incubation at 30 °C and 225 rpm agitation for 3 days in 24-well culture plates. Main fermentation samples were centrifuged to precipitate expression host cells and the supernatant was sterile filtered at 0.2 μm. The filtrate was used directly for lipase enzymatic activity characterization without further treatment. The activity was determined in tributyrin/agarose emulsion, for example as described below for Example 2 or by IDF method. In particular, the IDF method was used for dosing in Examples 3-6.

### Example 2—Enzymatic Activity on Triacylglycerols (TAG) Substrates

The enzymatic activity of each microbial lipase herein disclosed (SEQ ID NO: 1-11) towards short chain fatty acids (C₄, substrate tributyrin), as well as long chain fatty acids (C₁₆ and C₁₈, substrate olive oil), in 96-well plate assays, was performed as follows below. The same procedure (of determining the enzymatic activity towards short chain fatty acids (C₄, substrate tributyrin)) and long chain fatty acids (C₁₆ and C₁₈, substrate olive oil), in 96-well plate assays) was also conducted for a commercial microbial lipase from Rhizomucour miehei (Palatase®, Palatase® 20000 L, ≥20,000 U/g available from Novozymes A/S). The commercial microbial lipase from Rhizomucour miehei is herein labelled as commercial microbial lipase A.

Enzymatic activity for short chain fatty acids in plate: a tributyrin/agarose emulsion was generated by dissolving 1.4% low-melting agarose in 250 mM sodium acetate buffer pH 5.25 including 40 mM CaCl₂), addition of 1% (v/v) tributyrin to the solubilized agarose at 60 °C, and subsequent emulsification for 2 min using an IKA T25 Ultra-Turrax emulsifier at speed 13.5. A total volume of 50 μL emulsion was transferred to each well of a transparent 96-well assay plate and allowed to solidify at room temperature. Lipase reaction was started by adding 20 μL enzyme on top of the emulsion layer, in particular wherein 20 μL enzyme correspond to 20 μL of filtrate obtained from Example 1. Clearance of turbidity of the emulsion by enzymatic hydrolysis of substrate tributyrin was followed by measuring absorbance at 600 nm in a plate reader. Each assay was calibrated with various dilutions of commercial microbial lipase A with known enzymatic activity.

Enzymatic activity for long chain fatty acids in plate: an olive oil/agarose emulsion was generated by dissolving 1.4% low-melting agarose in 250 mM sodium acetate buffer pH 5.25 including 40 mM CaCl₂), addition of 2.5% (v/v) olive oil and 0.001% rhodamine B (w/v) to the solubilized agarose at 60 °C, and subsequent emulsification for 2 min using an IKA T25 Ultra-Turrax emulsifier at speed 13.5. A total volume of 150 μL emulsion was transferred to each well of a non-transparent black 96-well assay plate and allowed to solidify at room temperature. Lipase reaction was started by adding 30 μL enzyme on top of the emulsion layer, in particular wherein 30 μL enzyme correspond to 30 μL of filtrate obtained from Example 1. Binding of rhodamine B to long chain fatty acids released by enzymatic hydrolysis of substrate olive oil was followed by measuring fluorescence at 355 nm excitation and 590 nm emission in a plate reader. Each assay was calibrated with various dilutions of commercial microbial lipase A with known enzymatic activity.

Short chain fatty acid specificity C₄/C₁₆-C₁₈ was determined, for each microbial lipase herein disclosed, by calculating the ratio of hydrolytic activities on substrate tributyrin (C₄) and olive oil (C₁₆-C₁₈).

The ratio C₄/C₁₆-C₁₈ of each lipase (SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 and/or SEQ ID NO: 11) is normalized for the ratio C₄/C₁₆-C₁₈ of the microbial commercial lipase (A) used. Table 1 shows that the microbial lipases herein disclosed (SEQ ID NO: 1-11) have a higher specificity for the short chain fatty acid butyric acid (C₄) as compared to commercial microbial lipase A, in particular the microbial lipases of herein disclosed (SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 and/or SEQ ID NO: 11) are 2-770 times more C₄-fatty acid specific than the commercial microbial lipase A.

TABLE 1
Short chain fatty acid specificity (C₄/C₁₆-C₁₈) determined
with TAG substrates tributyrin and olive oil of lipases with
SEQ ID NO: 1, SEQ ID NO: 2, SEQ ID NO: 3, SEQ ID NO: 4, SEQ
ID NO: 5, SEQ ID NO: 6, SEQ ID NO: 7, SEQ ID NO: 8, SEQ ID
NO: 9, SEQ ID NO: 10, SEQ ID NO: 11 and commercial microbial
lipase A and respective improvement factor (IF4).

| | | IF4 = (C₄/C₁₆-C₁₈)_{A or SEQ ID NO: 1-11}/ |
|---|---|---|
| | C₄/C₁₆-C₁₈ | (C₄/C₁₆-C₁₈)_{A} |
| A | 1.2 | 1.0 |
| 1 | 101.0 | 83.3 |
| 2 | 5.0 | 4.1 |
| 3 | 770.5 | 635.3 |
| 4 | 7.4 | 6.1 |
| 5 | 17.2 | 14.2 |
| 6 | 2.3 | 1.9 |
| 7 | 8.0 | 6.6 |
| 8 | 14.0 | 11.6 |
| 9 | 14.5 | 11.9 |
| 10 | 6.1 | 5.0 |
| 11 | 2.4 | 2.0 |

### Example 3—Enzymatic Activity in Cream

The lipases herein disclosed (SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 or SEQ ID NO: 11), were additionally analyzed for their enzymatic activity towards milk fat and the released free fatty acids were quantified (Table 2). The same procedure was conducted for a commercial microbial lipase (commercial microbial lipase A) from Rhizomucour miehei (Palatase®) available from Novozymes A/S.

Lipase activity on milk fat in cream was carried out as follows. Fresh whipping cream (38% fat) and equal amounts of water were mixed by stirring for 30 min. Enzymatic reaction was started by adding 20 UL lipase (from a 4000 LFU/L stock solution) to 1 mL cream/water mix and proceeded at 37 °C for 20 hours, in particular this dosage of enzyme applies both to the commercial microbial lipase A and to lipases of SEQ ID NO: 1-11. Subsequently, free fatty acids were extracted from the reaction and quantified by gas chromatography-mass spectrometry (GC-MS), using standard protocols in the art for quantifying fatty acids. Alternatively the disclosure of Jong C., de and Badings H. T. in J. High Resolution Chromatography. 13, 84-98 (1990) can also be used.

Obtained fatty acid quantities of lipase reactions were corrected for the respective quantities determined for a similar cream sample without added lipase.

Subsequently, specificity for butyric acid (C_{4:0}), and caproic acid (C_{6:0}), caprylic acid (C_{8:0}) and capric acid (C_{10:0}) and the respective improvement factors compared to commercial microbial lipase A were determined based on the obtained data from lipolysis in cream (Table 2). Short chain fatty acid specificities (C₄/C₁₆-C₁₈; Co/C₁₆-C₁₈) were obtained by calculating the ratio of the molar fraction of C_{4:0} or C_{6:0} and the sum of molar fractions of C_{16:0}, C_{18:0}, C_{18:1} and C_{18:2}. Improvement factors IF4 and IF6 were calculated by dividing specificity values for C_{4:0} and C_{6:0} by the respective values of commercial lipase A. Medium chain fatty acid specificities (C₈/C₁₆-C₁₈; C₁₀/C₁₆-C₁₈) were obtained by calculating the ratio of the molar fraction of C_{8:0} or C_{10:0} and the sum of molar fractions of C_{16:0}, C_{18:0}, C_{18:1} and C_{18:2}. Improvement factors IF8 and IF10 were calculated by dividing specificity values for C_{8:0} and C_{10:0}, respectively, by the respective values of commercial lipase A.

The ratio C₄/C₁₆-C₁₈ of each lipolytic lipase is determined by diving C_{4:0} by the sum of C_{16:0}, C_{18:0}, C_{18:1} and C_{18:2} of the respective lipolytic lipase. The ratio C₄/C₁₆-C₁₈ is indicative of the specificity of the respective lipase for the release of butyric acid (C₄) from milk fat, relative to the release of fatty acids with chain lengths C₁₆-C₁₈.

The ratio Co/C₁₆-C₁₈ of each lipolytic lipase is determined by diving C_{6:0} by the sum of C_{16:0}, C_{18:0}, C_{18:1} and C_{18:2} of the respective lipolytic lipase. The ratio C₆/C₁₆-C₁₈ is indicative of the specificity of the respective lipase for the release of caproic acid (C₆) from milk fat, relative to the release of fatty acids with chain lengths C₁₆-C₁₈.

The ratio C₈/C₁₆-C₁₈ of each lipolytic lipase is determined by diving C_{8:0} by the sum of C_{16:0}, C_{18:0}, C_{18:1} and C_{18:2} of the respective lipolytic lipase. The ratio C₈/C₁₆-C₁₈ is indicative of the specificity of the respective lipase for the release of caproic acid (C₈) from milk fat, relative to the release of fatty acids with chain lengths C₁₆-C₁₈.

The ratio C₁₀/C₁₆-C₁₈ of each lipolytic lipase is determined by diving C_{10:0} by the sum of C_{16:0}, C_{18:0}, C_{18:1} and C_{18:2} of the respective lipolytic lipase. The ratio C₁₀/C₁₆-C₁₈ is indicative of the specificity of the respective lipase for the release of caproic acid (C₁₀) from milk fat, relative to the release of fatty acids with chain lengths C₁₆-C₁₈.

TABLE 2
Molar fractions of fatty acids released in cream by commercial
microbial lipase A and SEQ ID NO: 1, SEQ ID NO: 2, SEQ ID NO:
3, SEQ ID NO: 4, SEQ ID NO: 5, SEQ ID NO: 6, SEQ ID NO: 7, SEQ
ID NO: 8, SEQ ID NO: 9, SEQ ID NO: 10 and SEQ ID NO: 11.

| | C_{4:0} | C_{6:0} | C_{8:0} | C_{10:0} | C_{12:0} | C_{14:0} | C_{16:0} | C_{18:0} | C_{18:1} | C_{18:2} |
|---|---|---|---|---|---|---|---|---|---|---|
| A | 1.2 | 0.5 | 0.9 | 1.2 | 2.2 | 10.3 | 41.7 | 21.4 | 19.5 | 1.1 |
| 1 | 82.5 | 5.4 | 1.3 | 1.2 | 0.5 | 1.3 | 3.1 | 1.7 | 2.9 | 0.1 |
| 2 | 15.3 | 2.6 | 5.5 | 7.8 | 3.2 | 3.9 | 21.4 | 8.8 | 31.5 | 0.0 |
| 3 | 87.6 | 2.2 | 1.0 | 0.3 | 0.6 | 1.8 | 3.3 | 1.5 | 1.5 | 0.1 |
| 4 | 15.6 | 4.2 | 4.3 | 0.0 | 4.2 | 3.2 | 19.6 | 17.3 | 18.6 | 13.0 |
| 5 | 11.0 | 8.7 | 2.8 | 0.9 | 2.8 | 9.8 | 32.3 | 13.8 | 15.7 | 2.3 |
| 6 | 3.1 | 4.7 | 5.8 | 1.8 | 5.8 | 4.6 | 21.7 | 17.3 | 0.0 | 6.3 |
| 7 | 8.0 | 1.6 | 0.6 | 1.6 | 2.1 | 9.4 | 39.1 | 14.6 | 21.1 | 1.8 |
| 8 | 17.5 | 0.0 | 2.3 | 3.8 | 3.6 | 4.3 | 19.9 | 14.3 | 32.8 | 1.4 |
| 9 | 31.3 | 4.1 | 1.6 | 0.6 | 3.5 | 16.1 | 28.8 | 3.8 | 10.1 | 0.0 |
| 10 | 18.0 | 24.2 | 9.5 | 7.5 | 3.6 | 6.9 | 8.0 | 6.3 | 14.2 | 1.8 |
| 11 | 22.4 | 8.5 | 9.8 | 12.9 | 4.3 | 4.3 | 5.8 | 3.5 | 27.4 | 1.1 |

TABLE 3
Short chain and medium chain fatty acid specificities (C₄/
C₁₆-C₁₈, C₆/C₁₆-C₁₈, C₈/C₁₆-C₁₈, C₁₀/C₁₆-C₁₈) and improvement
factors (IF4, IF6, IF8, IF10) in cream by commercial microbial
lipase A and SEQ ID NO: 1, SEQ ID NO: 2, SEQ ID NO: 3, SEQ
ID NO: 4, SEQ ID NO: 5, SEQ ID NO: 6, SEQ ID NO: 7, SEQ ID
NO: 8, SEQ ID NO: 9, SEQ ID NO: 10 and SEQ ID NO: 11.

| | C₄/ | C₆/ | C₈/ | C₁₀/ | | | | |
|---|---|---|---|---|---|---|---|---|
| | C₁₆- | C₁₆- | C₁₆- | C₁₆- | | | | |
| | C₁₈ | C₁₈ | C₁₈ | C₁₈ | IF4 | IF6 | IF8 | IF10 |
| A | 0.014 | 0.006 | 0.010 | 0.014 | 1.0 | 1.0 | 1.0 | 1.0 |
| 1 | 10.645 | 0.697 | 0.165 | 0.159 | 744.1 | 113.1 | 16.1 | 11.0 |
| 2 | 0.249 | 0.043 | 0.063 | 0.086 | 17.4 | 6.9 | 6.2 | 5.9 |
| 3 | 13.600 | 0.334 | 0.155 | 0.053 | 950.7 | 54.2 | 15.2 | 3.7 |
| 4 | 0.227 | 0.062 | 0.062 | 0.000 | 15.9 | 10.0 | 6.1 | 0.0 |
| 5 | 0.172 | 0.136 | 0.043 | 0.015 | 12.0 | 22.0 | 4.2 | 1.0 |
| 6 | 0.711 | 0.103 | 0.129 | 0.040 | 49.7 | 16.7 | 12.6 | 2.8 |
| 7 | 0.105 | 0.021 | 0.008 | 0.021 | 7.3 | 3.4 | 0.8 | 1.4 |
| 8 | 0.255 | 0.000 | 0.033 | 0.056 | 17.9 | 0.1 | 3.2 | 3.9 |
| 9 | 0.733 | 0096 | 0.039 | 0.015 | 51.2 | 15.6 | 3.8 | 1.0 |
| 10 | 0.594 | 0.797 | 0.314 | 0.247 | 41.5 | 129.2 | 30.7 | 17.0 |
| 11 | 0.595 | 0.226 | 0.261 | 0.342 | 41.6 | 26.6 | 25.5 | 23.7 |

All the microbial lipolytic enzymes herein disclosed (SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 and/or SEQ ID NO: 11) show an IF4 higher than 1, in cream, meaning they have a higher specificity for the short chain fatty acid butyric acid (C_{4:0}) compared to commercial lipase A (Table 3) and can, therefore, be successfully used in a process for producing a food product wherein the presence of short chain fatty acid butyric acid (C_{4:0}) is needed and desired, such as for reducing soapiness and increase in butyric flavors in a food product.

Microbial lipolytic enzymes with SEQ ID NO: 1-7 and SEQ ID NO: 9-11 show an IF6 higher, than 1 in cream, meaning they have a higher specificity for the short chain fatty acid caproic acid (C_{6:0}) compared to commercial lipase A.

Microbial lipolytic enzymes with SEQ ID NO: 1-6 and SEQ ID NO: 8-11 show an IF8 higher, than 1 in cream, meaning they have a higher specificity for the medium chain fatty acid caprylic acid (C_{8:0}) compared to commercial lipase A.

Microbial lipolytic enzymes with SEQ ID NO: 1-3, SEQ ID NO: 6-8 and SEQ ID NO: 10-11 show an IF10 higher, than 1 in cream, meaning they have a higher specificity for the medium chain fatty acid capric acid (C_{10:0}) compared to commercial lipase A.

Microbial lipolytic enzyme with SEQ ID NO: 1 has a higher specificity for C_{4:0} than for any other fatty acid, in cream. The same applies for microbial lipolytic enzymes having SEQ ID NO: 3 or SEQ ID NO: 9 (Table 2).

### Example 4—Enzymatic Activity in Cheese

SEQ ID NO: 1 was further tested in cheese, in particular in Feta cheese and in Provolone cheese (Table 4). Example 4 was carried out with an identical dosage of enzyme (commercial microbial lipase A and SEQ ID NO: 1), in particular with a dosage of about 1.7 LFU/L of enzyme per liter of milk was used for the production of Feta cheese and between 5-10× for the Provolone cheese. The dosage was determined by the IDF method.

Microbial lipase SEQ ID NO: 1 was added during the cheese making process. The cheese making process of, for example Feta cheese or Provolone cheese, is well known in the art and to the skilled person. Subsequently, free fatty acids were extracted from cheeses after 1 month of ripening and quantified by GC-MS or as disclosed in Example 3. Obtained fatty acid quantities of lipase reactions were corrected for the respective quantities determined for a similar cheese without added lipase. Short chain fatty acid specificities were obtained by calculating the ratio of the molar fraction of C_{4:0} or C_{6:0} and the sum of molar fractions of C_{16:0}, C_{18:0}, C_{18:1} and C_{18:2}. Improvement factors IF4 and IF6 were calculated by dividing specificity values for C_{4:0} and C_{6:0} by the respective values of commercial lipase A.

TABLE 4
Molar fractions of fatty acids released in Feta cheese and Provolone cheese by commercial
microbial lipase A and SEQ ID NO: 1, after 1-month of ripening, as well as short chain fatty
acid specificities (C₄/C₁₆-C₁₈ and C₆/C₁₆-C₁₈) and improvement factors (IF4 and IF6).

| | | | | | | | | | | | C₄/ | C₆/ | | |
|---|---|---|---|---|---|---|---|---|---|---|---|---|---|---|
| | | | | | | | | | | | C₁₆- | C₁₆- | | |
| | C_{4:0} | C_{6:0} | C_{8:0} | C_{10:0} | C_{12:0} | C_{14:0} | C_{16:0} | C_{18:0} | C_{18:1} | C_{18:2} | C₁₈ | C₁₈ | IF4 | IF6 |
| | Feta cheese | | | | | | | | | | | | | |
| A | 2.0 | 0.8 | 2.3 | 4.6 | 6.2 | 14.6 | 35.2 | 9.4 | 22.5 | 2.5 | 0.029 | 0.012 | 1.0 | 1.0 |
| 1 | 50.3 | 5.5 | 0.0 | 3.7 | 6.3 | 5.5 | 12.3 | 0.0 | 11.2 | 0.0 | 2.135 | 0.231 | 74.5 | 19.9 |
| | Provolone cheese | | | | | | | | | | | | | |
| A | 8.7 | 2.1 | 2.2 | 3.6 | 4.9 | 11.6 | 28.6 | 7.0 | 27.2 | 4.0 | 0.130 | 0.032 | 1.0 | 1.0 |
| 1 | 82.8 | 3.4 | 1.4 | 1.1 | 1.0 | 0.9 | 3.1 | 1.4 | 2.1 | 0.7 | 11.402 | 0.468 | 87.7 | 14.8 |

SEQ ID NO: 1 has a higher specificity for C_{4:0}-fatty acids than commercial microbial lipase A, in Feta cheese and in Provolone cheese, and a higher specificity for C_{4:0}-fatty acids than for any other fatty acid, both Feta cheese and Provolone cheese, which is in line with the results obtained in cream (Example 3). Further, SEQ ID NO: 1 also has a higher specificity for C_{6:0}-fatty acids than commercial microbial lipase A, in Feta cheese and in Provolone cheese, which is also in line with the results obtained in cream (Example 3).

SEQ ID NO: 1 also shows a lower specificity for medium to long chain fatty acids such as C_{8:0}, C_{10:0}, C_{14:0}, C_{16:0}, C_{18:0}, C_{18:1} or C_{18:2} fatty acids when compared to commercial microbial lipase A in Feta cheese. Furthermore, SEQ ID NO: 1 also shows a lower specificity for medium to long chain fatty acids such as C_{8:0}, C_{10:0}, C_{12:0}, C_{14:0}, C_{16:0}, C_{18:0}, C_{18:1} or C_{18:2} fatty acids when compared to commercial microbial lipase A in Provolone cheese. These results are in line with the results obtained in cream (Example 3). Thus, a cheese produced using a microbial lipase comprising SEQ ID NO: 1 or wherein the microbial lipase is microbial lipase of SEQ ID NO: 1 is less soapy and more butyric than a cheese produced with a known microbial lipase. Identical results are expected for SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 and/or SEQ ID NO: 11.

Identical results are also expected for other types of cheese, such as Pecorino, Parmesan, Grana Padano, Parmigiano Reggiano, Romano, Chester, Danbo, Manchego, Saint Paulin, Cheddar, Monterey, Colby, Edam, Gouda, Muenster, Swiss type, Gruyere, Emmental; pasta filata cheeses such as Mozzarella, and Queso fresco cheese; fresh cheese such as Ricotta, Cream cheese, Neufchatel or Cottage cheese; cream cheese, white mold cheese such as Brie and Camembert cheese, blue mold cheese such as Gorgonzola and Danish blue cheese; processed cheese, cheese-like product, or an enzyme-modified cheese.

The microbial lipases herein disclosed are responsible for an increase in the C₄-fatty acid specificity in comparison with the prior art microbial lipases, thus leading to a reduced soapiness of cheese and an increase in the butyric flavor of cheese.

### Example 5—Sensorial Evaluation

A sensorial evaluation of cheese, in particular in Feta cheese aged 3-months, was also performed by rating the taste according to sensational parameters butyric and soapy on a scale from 1 (none) to 6 (intense). The Feta cheese was produced using identical conditions using a commercial A or SEQ ID NO: 1 or SEQ ID NO: 3 or SEQ ID NO: 5. The amount or dosage of enzyme (commercial microbial lipase A, SEQ ID NO: 1 or SEQ ID NO: 3 or SEQ ID NO: 5) used was 1.7 LFU/L of milk determined by the IDF method (Table 5).

TABLE 5
Sensorial evaluation carried out with Feta cheese
aged 3-months using of commercial microbial lipase
A, SEQ ID NO: 1, SEQ ID NO: 3 and SEQ ID NO: 5.

Feta cheese aged 3-months

| | Butyric | Soapy |
|---|---|---|
| A | 3 | 5 |
| 1 | 4 | 1 |
| 3 | 4 | 1 |
| 5 | 4 | 3 |

Table 5 shows a reduction of soapiness in Feta cheese, aged 3-months when SEQ ID NO: 1 or 3 or 5 is used when compared with commercial lipase A, while simultaneously an increase in the butyric flavor is observed for the same samples.

### Example 6—Enzymatic Activity in Cheese

SEQ ID NO: 1, 2, 3, 6, 8, 10 and 11 were further tested in cheese, in particular in Feta cheese. Example 6 was carried out with an identical dosage of enzyme (the commercial microbial lipase A and SEQ ID NO: 1, 2, 3, 6, 8, 10 and 11), in particular the a dosage of enzyme was of about 2.3 LFU/L of milk, determined by the IDF method, for the production of Feta cheese.

Microbial lipases SEQ ID NO: 1, 2, 3, 6, 8, 10 and 11 were individually added during the cheese making process. Subsequently, free fatty acids were extracted from cheeses after 1-month (Table 6) and 2-months (Table 8) of ripening and quantified by GC-MS or as disclosed in Example 3. Obtained fatty acid quantities of lipase reactions were corrected for the respective quantities determined for a similar cheese without added lipase. Short- and medium-chain fatty acid specificities were obtained by calculating the ratio of the molar fraction of C_{4:0}, C_{6:0}, C_{8:0} or C_{10:0} and the sum of molar fractions of C_{16:0}, C_{16:1}, C_{18:0}, C_{18:1}, C_{18:2} and C_{18:3} (Tables 7 and 9). Improvement factors IF4, IF6, IF8 and IF10 were calculated by dividing specificity values for C_{4:0}, C_{6:0} C_{8:0} or C_{10:0} by the respective values of commercial lipase A (Tables 7 and 9).

TABLE 6
Molar fractions of fatty acids released in Feta cheese by commercial
microbial lipase A and SEQ ID NO: 1, 2, 3, 6, 10 and 11, after
1-monthh of ripening. All lipases were dosage at 2.3 LFU/L.

| | C_{4:0} | C_{6:0} | C_{8:0} | C_{10:0} | C_{12:0} | C_{14:0} | C_{16:0} | C_{16:1} | C_{18:0} | C_{18:1} | C_{18:2} | C_{18:3} |
|---|---|---|---|---|---|---|---|---|---|---|---|---|
| A | 4.3 | 3.1 | 8.5 | 15.6 | 12.7 | 15.4 | 24.8 | 1.5 | 4.7 | 8.2 | 0.8 | 0.4 |
| 1 | 84.2 | 3.8 | 2.1 | 2.3 | 1.7 | 1.4 | 2.2 | 0.2 | 0.9 | 0.9 | 0.2 | 0.0 |
| 2 | 22.8 | 17.6 | 9.1 | 9.6 | 3.9 | 5.7 | 15.4 | 0.8 | 5.2 | 8.7 | 0.9 | 0.3 |
| 3 | 88.0 | 6.7 | 1.5 | 0.9 | 1.9 | 0.7 | 0.0 | 0.3 | 0.0 | 0.0 | 0.0 | 0.0 |
| 6 | 5.2 | 8.7 | 15.8 | 17.7 | 9.9 | 10.3 | 15.1 | 1.2 | 5.4 | 9.1 | 1.1 | 0.4 |
| 10 | 15.9 | 26.7 | 18.2 | 14.6 | 5.4 | 5.4 | 5.7 | 0.8 | 1.6 | 5.0 | 0.5 | 0.2 |
| 11 | 24.9 | 9.0 | 13.2 | 19.6 | 5.8 | 4.9 | 6.5 | 1.2 | 1.1 | 12.1 | 1.3 | 0.5 |

TABLE 7
Short- and medium-chain fatty acid specificities (C₄/C₁₆-
C₁₈, C₆/C₁₆-C₁₈, C₈/C₁₆-C₁₈, C₁₀/C₁₆-C₁₈) and improvement
factors (IF4, IF6, IF8, IF10) in Feta cheese by commercial
microbial lipase A and SEQ ID NO: 1, 2, 3, 6, 10 and 11, after
1-monthh of ripening. All lipases were dosage at 2.3 LFU/L.

| | C₄/ | C₆/ | C₈/ | C₁₀/ | | | | |
|---|---|---|---|---|---|---|---|---|
| | C₁₆- | C₁₆- | C₁₆- | C₁₆- | | | | |
| | C₁₈ | C₁₈ | C₁₈ | C₁₈ | IF4 | IF6 | IF8 | IF10 |
| A | 0.107 | 0.076 | 0.211 | 0.386 | 1.0 | 1.0 | 1.0 | 1.0 |
| 1 | 18.733 | 0.837 | 0.478 | 0.511 | 175.7 | 11.1 | 2.3 | 1.3 |
| 2 | 0.730 | 0.564 | 0.291 | 0.307 | 6.8 | 7.5 | 1.4 | 0.8 |
| 3 | 291.602 | 22.318 | 4.936 | 2.953 | 2734.9 | 294.8 | 23.4 | 7.7 |
| 6 | 0.160 | 0.270 | 0.489 | 0.546 | 1.5 | 3.6 | 2.3 | 1.4 |
| 10 | 1.158 | 1.943 | 1.327 | 1.060 | 10.9 | 25.7 | 6.3 | 2.7 |
| 11 | 1.097 | 0.397 | 0.583 | 0.864 | 10.3 | 5.2 | 2.8 | 2.2 |

All the tested microbial lipolytic enzymes show an IF4 higher than 1, in Feta cheese, after 1-month of ripening. Thus, the tested enzymes have a higher specificity for the short chain fatty acid butyric acid (C_{4:0}) compared to commercial lipase A (Table 7) and can, therefore, be successfully used in a process for producing a food product wherein the presence of short chain fatty acid butyric acid (C_{4:0}) is needed and desired, such as for reducing soapiness and increase in butyric flavors in a food product. This observation, of an IF higher than 1 for the tested lipases, is in line with the results of Example 3, where data was obtained in cream. Further, specifically for SEQ ID NO: 1, this observation is also in line with the results of Example 4.

Further, these enzymes show an IF6, IF8 and IF10 higher than 1, in Feta cheese, after 1-month of ripening, meaning they have a higher specificity for the short chain fatty acid caproic acid (C_{6:0}) and for medium chain fatty acid caprylic acid (C_{8:0}) and capric acid (C_{10:0}) compared to commercial lipase A, with the exception of SEQ ID NO: 2 which has a IF10 below 1. The results obtained for these sequences (SEQ ID NO: 1, 2, 3, 6, 10 and 11) and their specificity for C_{6:0}-fatty acids, C_{8:0}-fatty acids and C_{10:0}-fatty acids are essentially in line with the results obtained in cream (Example 3).

In conclusion, the results obtained in cheese, after 1-month of ripening (Example 4 and 6) are in line with the results obtained in cream (Example 3), showing that the results obtained in cream can be used to plausibly extrapolate the behavior of the non-tested sequences from cream to cheese.

TABLE 8
Molar fractions of fatty acids released in Feta cheese by commercial
microbial lipase A and SEQ ID NO: 1, 2, 3, 6, 8, 10 and 11, after
2-monthh of ripening. All lipases were dosage at 2.3 LFU/L.

| | C_{4:0} | C_{6:0} | C_{8:0} | C_{10:0} | C_{12:0} | C_{14:0} | C_{16:0} | C_{16:1} | C_{18:0} | C_{18:1} | C_{18:2} | C_{18:3} |
|---|---|---|---|---|---|---|---|---|---|---|---|---|
| A | 4.3 | 2.3 | 9.3 | 17.3 | 13.7 | 14.3 | 20.1 | 1.5 | 4.2 | 10.9 | 1.4 | 0.7 |
| 1 | 69.4 | 5.6 | 5.3 | 6.4 | 2.3 | 2.8 | 4.0 | 0.4 | 0.3 | 3.1 | 0.3 | 0.1 |
| 2 | 17.8 | 17.5 | 9.6 | 13.6 | 5.0 | 6.3 | 15.4 | 0.8 | 5.0 | 7.9 | 0.8 | 0.3 |
| 3 | 94.2 | 0.9 | 2.0 | 0.7 | 0.0 | 0.0 | 0.8 | 0.0 | 1.0 | 0.4 | 0.0 | 0.0 |
| 6 | 6.5 | 4.8 | 11.2 | 15.9 | 11.2 | 12.7 | 17.4 | 1.5 | 5.0 | 12.0 | 1.4 | 0.6 |
| 8 | 36.6 | 14.2 | 9.1 | 12.7 | 3.8 | 5.6 | 9.8 | 0.6 | 2.4 | 4.5 | 0.6 | 0.3 |
| 10 | 16.0 | 27.9 | 18.6 | 17.0 | 5.3 | 5.4 | 3.4 | 0.6 | 0.7 | 4.6 | 0.4 | 0.2 |
| 11 | 32.7 | 7.5 | 13.2 | 22.0 | 4.6 | 4.0 | 3.7 | 0.8 | 1.0 | 9.3 | 0.8 | 0.4 |

TABLE 9
Short- and medium-chain fatty acid specificities (C₄/C₁₆-C₁₈, C₆/
C₁₆-C₁₈, C₈/C₁₆-C₁₈, C₁₀/C₁₆-C₁₈) and improvement
factors (IF4, IF6, IF8, IF10) in Feta cheese by commercial
microbial lipase A and SEQ ID NO: 1, 2, 3, 6, 8, 10 and 11,
after 2-monthh of ripening. All lipases were dosage at 2.3 LFU/L.

| | C₄/ | C₆/ | C₈/ | C₁₀/ | | | | |
|---|---|---|---|---|---|---|---|---|
| | C₁₆- | C₁₆- | C₁₆- | C₁₆- | | | | |
| | C₁₈ | C₁₈ | C₁₈ | C₁₈ | IF4 | IF6 | IF8 | IF10 |
| A | 0.112 | 0.059 | 0.239 | 0.444 | 1.0 | 1.0 | 1.0 | 1.0 |
| 1 | 8.357 | 0.669 | 0.635 | 0.776 | 74.8 | 11.2 | 2.7 | 1.7 |
| 2 | 0.588 | 0.577 | 0.319 | 0.449 | 5.3 | 9.7 | 1.3 | 1.0 |
| 3 | 42.932 | 0.410 | 0.908 | 0.304 | 384.3 | 6.9 | 3.8 | 0.7 |
| 6 | 0.172 | 0.126 | 0.296 | 0.420 | 1.5 | 2.1 | 1.2 | 0.9 |
| 8 | 2.024 | 0.785 | 0.504 | 0.705 | 18.1 | 13.2 | 2.1 | 1.6 |
| 10 | 1.607 | 2.807 | 1.870 | 1.707 | 14.4 | 47.2 | 7.8 | 3.8 |
| 11 | 2.044 | 0.469 | 0.829 | 1.377 | 18.3 | 7.9 | 3.5 | 3.1 |

All the tested microbial lipolytic enzymes show an IF4 higher than 1, in Feta cheese, after 2-month of ripening. Thus, the tested enzymes have a higher specificity for the short chain fatty acid butyric acid (C_{4:0}) compared to commercial lipase A (Table 7) and can, therefore, be successfully used in a process for producing a food product wherein the presence of short chain fatty acid butyric acid (C_{4:0}) is needed and desired, such as for reducing soapiness and increase in butyric flavors in a food product.

Microbial lipolytic enzymes with SEQ ID NO: 1, 2, 3, 6, 8, 10 and 11 show an IF6 and IF8 higher than 1, meaning they have a higher specificity for the short chain fatty acid caproic acid (C_{6:0}) and for medium chain fatty acid caprylic acid (C_{8:0}) compared to commercial lipase A.

The tested enzymes, with the exception of SEQ ID NO: 3, also shown an IF10 higher than 1, and have, therefore, a higher specificity for medium chain fatty capric acid (C_{10:0}) compared to commercial lipase A.

Further and as previously mentioned this invention relates to flavor enhancement but also with shortening of the ripening times for ripened cheeses. The comparison between free fatty acid profiles after 1-month or 2-months of ripening shows that it is possible to successfully reduce ripening time from 2-months (or more) to 1-month (or less), while the cheese still presents the desired short-chain fatty acid profile.

Identical results to those disclosed in Example 6 are also expected for other types of cheese, such as Pecorino, Parmesan, Grana Padano, Parmigiano Reggiano, Romano, Chester, Danbo, Manchego, Saint Paulin, Cheddar, Monterey, Colby, Edam, Gouda, Muenster, Swiss type, Gruyere, Emmental; pasta filata cheeses such as Mozzarella, and Queso fresco cheese; fresh cheese such as Ricotta, Cream cheese, Neufchatel or Cottage cheese; cream cheese, white mold cheese such as Brie and Camembert cheese, blue mold cheese such as Gorgonzola and Danish blue cheese; processed cheese, cheese-like product, or an enzyme-modified cheese.

In conclusion, the present invention discloses microbial wild-type lipases with a higher specificity for short chain fatty acids, specially C₄-fatty acid, than the known prior art microbial lipases, leading to a reduction in soapiness and an increase in butyric flavors of a food product such as cheese. Therefore, this invention provides microbial wild-type lipases which fulfill the vegetarian and/or kosher requirements, while simultaneously showing higher specificity for short chain fatty acids, specially C₄-fatty acid and lower specificity for medium to long chain fatty acids from milk fat and/or other fat, specially showing lower specificity for long chain fatty acids from milk fat and/or other fat, thereby avoiding an extremely unpleasant soapy taste in a food product, like cheese.

The present invention can be applied to other kinds of food products wherein the purpose is to reduce the unpleasant soapy taste of said food product.

Finally, if needed, the ripening time of the food product can be reduced from 2-months to 1-month, in particular in Feta cheese, while still maintaining the proper and desired short-chain fatty acid profile.

The use of the terms “a” and “an” and “the” and similar references in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### List of Preferred Embodiments in a Claim Format

1. Lipase comprising an amino acid sequence
   having at least 90% identity with SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11, preferably SEQ ID NO: 1 or SEQ ID NO: 3 or SEQ ID NO: 9 or SEQ ID NO: 11, more preferably SEQ ID NO: 1 or SEQ ID NO: 3 or SEQ ID NO: 11, even more preferably SEQ ID NO: 1, or
   having at least 95%, 96%, 97%, 98% or 99% identity with SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10 or SEQ ID NO: 11, preferably SEQ ID NO: 1 or SEQ ID NO: 3 or SEQ ID NO: 9 or SEQ ID NO: 11, more preferably SEQ ID NO: 1 or SEQ ID NO: 3 or SEQ ID NO: 11, even more preferably SEQ ID NO: 1, or
   wherein the sequence is SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10 or SEQ ID NO: 11, preferably SEQ ID NO: 1, preferably SEQ ID NO: 1 or SEQ ID NO: 3 or SEQ ID NO: 9 or SEQ ID NO: 11, more preferably SEQ ID NO: 1 or SEQ ID NO: 3 or SEQ ID NO: 11, even more preferably SEQ ID NO: 1.
2. Lipase according to the previous item, wherein said lipase has a higher specificity towards the release of C₄-fatty acids from a dairy composition comprising milk fat and/or other fat if compared with the release of C₁₀-fatty acids.
3. Lipase according to any of the previous items, wherein said lipase has a higher specificity towards the release of C₄-fatty acids from a dairy composition comprising milk fat and/or other fat if compared with the release of C₈-fatty acids, or if compared with the release of C₆-fatty acids, or if compared with the release of C₁₂-fatty acids or if compared with the release of C_{18:2}-fatty acids or if compared with the release of C₁₄-fatty acids or if compared with the release of C_{18:0}-fatty acids or if compared with the release of C_{18:1}-fatty acids or if compared with the release of C₁₆-fatty acids, or if compared with the release of C₈-fatty acids-C_{18:2}-fatty acids, or if compared with the release of C₁₀-fatty acids-C_{18:1}-fatty acids, or if compared with the release of C₁₂-fatty acids-C_{18:0}-fatty acids, or if compared with the release of C₁₄-fatty acids-C_{16:0}-fatty acids or if compared with the release of C₁₆-C₁₈-fatty acids.
4. Lipase according to any of the previous items, wherein said lipase has a higher specificity towards the release of C₄-fatty acids from a dairy composition comprising milk fat and/or other fat if compared with the release of C₁₀-fatty acids at a pH below 7, preferably 6.6-6.8, or at a pH below 6, preferably at a pH between 3.8-5.6, more preferably at a pH between 4.4-5.4, even more preferably at a pH between 4.6-5.2, or if compared with the release of C₆-fatty acids or if compared with the release of C₈-fatty acids or if compared with the release of C₁₂-fatty acids or if compared with the release of C₁₄-fatty acids or if compared with the release of C₁₆-fatty acids or if compared with the release of C_{18:1}-fatty acids or if compared with the release of C_{18:2}-fatty acids or if compared with the release of C_{18:3}-fatty acids or if compared with the release of C₁₆-C₁₈-fatty acids.
5. Lipase according to any of the previous items, wherein said wherein said lipase has a higher specificity towards the release of C₄-fatty acids from a dairy composition comprising milk fat and/or other fat if compared with the release of C₁₀-fatty acids at a temperature below 20 °C, preferably below 15 °C, more preferably below 10 °C, even more preferably between 5-8 °C, or if compared with the release of C₆-fatty acids or if compared with the release of C₈-fatty acids or if compared with the release of C₁₂-fatty acids or if compared with the release of C₁₄-fatty acids or if compared with the release of C₁₆-fatty acids or if compared with the release of C_{18:1}-fatty acids or if compared with the release of C_{18:2}-fatty acids or if compared with the release of C_{18:3}-fatty acid or if compared with the release of C₁₆-C₁₈-fatty acids.
6. Lipase according to any of the previous items, wherein said lipase is a microbial lipase, preferably an isolated microbial lipase, a recombinant microbial lipase or a synthetic microbial lipase.
7. Isolated DNA sequence or recombinant DNA sequence or synthetic DNA sequence encoding a lipase according to any of the preceding items, preferably wherein the isolated DNA sequence or recombinant DNA sequence or synthetic DNA is selected from a sequence having at least 90%, or at least 95%, or at least 96%, or at least 97%, or at least 98% or at least 99%, or 100% sequence identity with SEQ ID NO: 12 or SEQ ID NO: 13 or SEQ ID NO: 14 or SEQ ID NO: 15 or SEQ ID NO: 16 or SEQ ID NO: 17 or SEQ ID NO: 18 or SEQ ID NO: 19 or SEQ ID NO: 20 or SEQ ID NO: 21 or SEQ ID NO: 22 or SEQ ID NO: 23.
8. DNA sequence according to the preceding item further comprising a signal peptide sequence.
9. Vector comprising an isolated DNA sequence or recombinant DNA sequence or synthetic DNA sequence according to any of the previous items 7-8.
10. Host cell, preferably a recombinant host cell, comprising a sequence according to any of the previous items 1-8 or a vector according to previous item 9.
11. Host cell according to the previous item, wherein the host cell is selected from Pichia pastoris, Aspergillus or Bacillus subtilis, preferably Pichia pastoris or B. subtilis, more preferably Pichia pastoris.
12. Method for preparing a food product comprising a step of
   using a lipase comprising an amino acid sequence having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11, preferably having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 9 or SEQ ID NO: 11; or
   using a DNA sequence encoding a lipase, wherein the DNA sequence is selected from a sequence having at least 90%, or at least 95%, or at least 96%, or at least 97%, or at least 98% or at least 99%, or 100% sequence identity with SEQ ID NO: 12 or SEQ ID NO: 13 or SEQ ID NO: 14 or SEQ ID NO: 15 or SEQ ID NO: 16 or SEQ ID NO: 17 or SEQ ID NO: 18 or SEQ ID NO: 19 or SEQ ID NO: 20 or SEQ ID NO: 21 or SEQ ID NO: 22 or SEQ ID NO: 23, preferably having at least 90%, or at least 95%, or at least 96%, or at least 97%, or at least 98% or at least 99%, or 100% sequence identity with SEQ ID NO: 12 or SEQ ID NO: 13 or SEQ ID NO: 14 or SEQ ID NO: 20 or SEQ ID NO: 22 or SEQ ID NO: 23; preferably wherein the DNA sequence is an isolated DNA sequence or a recombinant DNA sequence or a synthetic DNA sequence, more preferably wherein the DNA sequence comprises a signal peptide sequence.
13. Method according to the previous item 12, wherein said lipase has a higher specificity towards the release of C₄-fatty acids from a dairy composition comprising milk fat and/or other fat if compared with the release of C₁₀-fatty acids or if compared with the release of C₈-fatty acids, or if compared with the release of C₆-fatty acids, or if compared with the release of C₁₂-fatty acids or if compared with the release of C_{18:2}-fatty acids or if compared with the release of C₁₄-fatty acids or if compared with the release of C_{18:0}-fatty acids or if compared with the release of C_{18:1}-fatty acids or if compared with the release of C₁₆-fatty acids, or if compared with the release of C₈-fatty acids-C_{18:2}-fatty acids, or if compared with the release of C₁₀-fatty acids-C_{18:1}-fatty acids, or if compared with the release of C₁₂-fatty acids-C_{18:0}-fatty acids, or if compared with the release of C₁₄-fatty acids-C_{16:0}-fatty acids or if compared with the release of C₁₆-C₁₈-fatty acids.
14. Method according to any of the previous items 12-13, wherein said lipase has a higher specificity towards the release of C₄-fatty acids from a dairy composition comprising milk fat and/or other fat if compared with the release of C₁₀-fatty acids at a pH below 7, preferably 6.6-6.8, or at a pH below 6, preferably at a pH between 3.8-5.6, more preferably at a pH between 4.4-5.4, even more preferably at a pH between 4.6-5.2, or if compared with the release of C₆-fatty acids or if compared with the release of C₈-fatty acids or if compared with the release of C₁₂-fatty acids or if compared with the release of C₁₄-fatty acids or if compared with the release of C₁₆-fatty acids or if compared with the release of C_{18:1}-fatty acids or if compared with the release of C_{18:2}-fatty acids or if compared with the release of C_{18:3}-fatty acid or if compared with the release of C₁₆-C₁₈-fatty acids.
15. Method according to any of the previous items 12-14, wherein said wherein said lipase has a higher specificity towards the release of C₄-fatty acids from a dairy composition comprising milk fat and/or other fat if compared with the release of C₁₀-fatty acids at a temperature below 20 °C, preferably below 15 °C, more preferably below 10 °C, even more preferably between 5-8 °C, or if compared with the release of C₆-fatty acids or if compared with the release of C₈-fatty acids or if compared with the release of C₁₂-fatty acids or if compared with the release of C₁₄-fatty acids or if compared with the release of C₁₆-fatty acids or if compared with the release of C_{18:1}-fatty acids or if compared with the release of C_{18:2}-fatty acids or if compared with the release of C_{18:3}-fatty acid or if compared with the release of C₁₆-C₁₈-fatty acids.
16. Method according to any of the previous items 12-15, wherein said lipase is a microbial lipase, preferably an isolated microbial lipase, a recombinant microbial lipase or a synthetic microbial lipase.
17. Method according to any of the previous items 12-16, wherein the food product is a dairy product selected from a cheese, or a processed cheese, or a cheese-like product, or an enzyme-modified cheese, or a butter, or a yogurt, or a cream, or a seasoning.
18. Method according to the previous items 12-17, wherein the dairy product is a cheese, preferably wherein the cheese is Feta cheese, or Provolone cheese, or Pecorino cheese, or Parmesan cheese, or Grana Padano cheese, or Parmigiano Reggiano cheese, or Romano cheese, or Chester cheese, or Danbo cheese, or Manchego cheese, or Saint Paulin cheese, or Cheddar cheese, or Monterey cheese, or Colby cheese, or Edam cheese, or Gouda cheese, or Muenster cheese, or Swiss type cheese, or Gruyere cheese, or Emmental cheese; or pasta filata cheeses such as Mozzarella, and Queso fresco cheese; or fresh cheese such as Ricotta, Cream cheese, Neufchatel or Cottage cheese; or cream cheese, or white mold cheese such as Brie and Camembert cheese, or blue mold cheese such as Gorgonzola and Danish blue cheese.
19. Method according to previous item 12, wherein the food product is a non-dairy product, preferably a non-dairy cheese or vegan cheese, preferably wherein the lipase has a higher specificity towards the release of C₄-fatty acids from a composition comprising fat such as vegetable fat or from a water-in-oil emulsion if compared with the release of C₁₀-fatty acids or if compared with the release of C₈-fatty acids, or if compared with the release of C₆-fatty acids, or if compared with the release of C₁₂-fatty acids or if compared with the release of C_{18:2}-fatty acids or if compared with the release of C₁₄-fatty acids or if compared with the release of C_{18:0}-fatty acids or if compared with the release of C_{18:1}-fatty acids or if compared with the release of C₁₆-fatty acids, or if compared with the release of C₈-fatty acids-C_{18:2}-fatty acids, or if compared with the release of C₁₀-fatty acids-C_{18:1}-fatty acids, or if compared with the release of C₁₂-fatty acids-C_{18:0}-fatty acids, or if compared with the release of C₁₄-fatty acids-C_{16:0}-fatty acids or if compared with the release of C₁₆-C₁₈-fatty acids.
20. Food product comprising a lipase, wherein the lipase comprises an amino acid sequence having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11, preferably having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 9 or SEQ ID NO: 11, and
   wherein the food product comprises more C₄-fatty acids than C₁₀-fatty acids or than any other fatty acid selected from C₆-fatty acids or C₈-fatty acids or C₁₂-fatty acids or C₁₄-fatty acids or C₁₆-fatty acids or C_{18:1}-fatty acids or C_{18:2}-fatty acids or C_{18:3}-fatty acids and/or
   wherein the food product comprises more C₆-fatty acids than C₁₀-fatty acids or than any other fatty acid selected from C₈-fatty acids or C₁₂-fatty acids or C₁₄-fatty acids or C₁₆-fatty acids or C_{18:1}-fatty acids or C_{18:2}-fatty acids or C_{18:3}-fatty acids.
21. Food product according to the previous item 20, wherein the food product is a dairy product selected from a cheese, a processed cheese, a cheese-like product, or a butter, a yogurt, a cream, a seasoning, preferably wherein the cheese is Feta cheese, or Provolone cheese, or Pecorino cheese, or Parmesan cheese, or Grana Padano cheese, or Parmigiano Reggiano cheese, or Romano cheese, or Chester cheese, or Danbo cheese, or Manchego cheese, or Saint Paulin cheese, or Cheddar cheese, or Monterey cheese, or Colby cheese, or Edam cheese, or Gouda cheese, or Muenster cheese, or Swiss type cheese, or Gruyere cheese, or Emmental cheese; or pasta filata cheeses such as Mozzarella, and Queso fresco cheese; or fresh cheese such as Ricotta, Cream cheese, Neufchatel or Cottage cheese; or cream cheese, or white mold cheese such as Brie and Camembert cheese, or blue mold cheese such as Gorgonzola and Danish blue cheese, more preferably Feta cheese or Provolone cheese; or wherein the food product is a non-dairy product such as a non-dairy cheese or vegan cheese.
22. Use of a lipase, for producing a food product,
   wherein the food product is a dairy product selected from a cheese, or a processed cheese, or an enzyme-modified cheese (EMC), or a cheese-like product, or a butter, or a yogurt, or a cream, or a seasoning, preferably for producing cheese, such as Feta cheese, or Provolone cheese, or Pecorino cheese, or Parmesan cheese, or Grana Padano cheese, or Parmigiano Reggiano cheese, or Romano cheese, or Chester cheese, or Danbo cheese, or Manchego cheese, or Saint Paulin cheese, or Cheddar cheese, or Monterey cheese, or Colby cheese, or Edam cheese, or Gouda cheese, or Muenster cheese, or Swiss type cheese, or Gruyere cheese, or Emmental cheese; or pasta filata cheeses such as Mozzarella, and Queso fresco cheese; or fresh cheese such as Ricotta, Cream cheese, Neufchatel or Cottage cheese; or cream cheese, or white mold cheese such as Brie and Camembert cheese, or blue mold cheese such as Gorgonzola and Danish blue cheese; more preferably the cheese is Feta cheese or Provolone cheese; or
   wherein the food product is a non-dairy product such as a non-dairy cheese or vegan cheese;
   and wherein the lipase comprises an amino acid sequence having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11, preferably having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 9 or SEQ ID NO: 11.
23. Use of a lipase for producing or releasing more C₄-fatty acids and/or C₆-fatty acids from a dairy composition comprising milk fat and/or other fat, such as vegetable fat, than for producing or releasing more C₈-fatty acids, C₁₀-fatty acids, C₁₂-fatty acids, C₁₄-fatty acids, C₁₆-fatty acids, C_{18:0}-fatty acids, C_{18:1}-fatty acids, or C_{18:2}-fatty acids, or C_{18:3}-fatty acids, wherein the lipase comprises an amino acid sequence having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11, preferably having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 9 or SEQ ID NO: 11, preferably wherein said lipase has a higher specificity towards the release of C₄-fatty acids from a dairy composition comprising milk fat and/or other fat if compared with the release of C₁₀-fatty acids or if compared with the release of C₈-fatty acids, or if compared with the release of C₆-fatty acids, or if compared with the release of C₁₂-fatty acids or if compared with the release of C_{18:2}-fatty acids or if compared with the release of C₁₄-fatty acids or if compared with the release of C_{18:0}-fatty acids or if compared with the release of C_{18:1}-fatty acids or if compared with the release of C₁₆-fatty acids, or if compared with the release of C₈-fatty acids-C_{18:2}-fatty acids, or if compared with the release of C₁₀-fatty acids-C_{18:1}-fatty acids, or if compared with the release of C₁₂-fatty acids-C_{18:0}-fatty acids, or if compared with the release of C₁₄-fatty acids-C_{16:0}-fatty acids or if compared with the release of C₁₆-C₁₈-fatty acids.
24. Use of a lipase for producing or releasing more C₄-fatty acids and/or C₆-fatty acids in a food product than for producing or releasing more C₈-fatty acids, C₁₀-fatty acids, C₁₂-fatty acids, C₁₄-fatty acids, C₁₆-fatty acids, C_{18:0}-fatty acids, C_{18:1}-fatty acids, or C_{18:2}-fatty acids, or C_{18:3}-fatty acids in the food product, wherein the lipase comprises an amino acid sequence having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11, preferably having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 9 or SEQ ID NO: 11,
   preferably wherein the food product is a dairy product selected from a cheese, or a processed cheese, or enzyme-modified cheese, or a cheese-like product, or a butter, or a yogurt, or a cream, or a seasoning, or
   preferably wherein the food product is a non-dairy product such as a non-dairy cheese or vegan cheese.
25. Use according to the previous item 24, wherein the food product is a dairy product selected from a cheese and wherein the cheese is Feta cheese, or Provolone cheese, or Pecorino cheese, or Parmesan cheese, or Grana Padano cheese, or Parmigiano Reggiano cheese, or Romano cheese, or Chester cheese, or Danbo cheese, or Manchego cheese, or Saint Paulin cheese, or Cheddar cheese, or Monterey cheese, or Colby cheese, or Edam cheese, or Gouda cheese, or Muenster cheese, or Swiss type cheese, or Gruyere cheese, or Emmental cheese; or pasta filata cheeses such as Mozzarella, and Queso fresco cheese; or fresh cheese such as Ricotta, Cream cheese, Neufchatel or Cottage cheese; or cream cheese, or white mold cheese such as Brie and Camembert cheese, or blue mold cheese such as Gorgonzola and Danish blue cheese, preferably Feta cheese or Provolone cheese.
26. Lipase consisting of an amino acid sequence having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4 or SEQ ID NO: 5 or SEQ ID NO: 6 or SEQ ID NO: 7 or SEQ ID NO: 8 or SEQ ID NO: 9 or SEQ ID NO: 10 or SEQ ID NO: 11, preferably having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 9 or SEQ ID NO: 11;
   wherein the lipase is expressed in Pichia pastoris and has a higher specificity towards the release of C₄-fatty acids from a dairy composition comprising milk fat and/or other fat if compared with the release of C₁₀-fatty acids, or if compared with the release of C₈-fatty acids, or if compared with the release of C₆-fatty acids, or if compared with the release of C₁₂-fatty acids or if compared with the release of C_{18:2}-fatty acids or if compared with the release of C₁₄-fatty acids or if compared with the release of C_{18:0}-fatty acids or if compared with the release of C_{18:1}-fatty acids or if compared with the release of C₁₆-fatty acids, or if compared with the release of C₈-fatty acids-C_{18:2}-fatty acids, or if compared with the release of C₁₀-fatty acids-C_{18:1}-fatty acids, or if compared with the release of C₁₂-fatty acids-C_{18:0}-fatty acids, or if compared with the release of C₁₄-fatty acids-C_{16:0}-fatty acids or if compared with the release of C₁₆-C₁₈-fatty acids.
27. Lipase according to item 26, wherein the lipase is used in any of the methods or uses herein disclosed.

### REFERENCES

### Non Patent Literature

Iwai, M. et al. Studies on Lipase II. Hydrolytic and esterifying actions of crystalline lipase of Aspergillus niger. J. Gen. Appl. Microbiol. 10, 13-22 (1964)
Oi, S. et al. Purification and Some Properties of a Fungal Lipase Preparation Used for Milk Flavouring. Agr. Biol. Chem. 33, 729-38 (1969)
Somkuti, G. A. et al. Lipase Activity of Mucor pusillus. Appl. Microbiol. 16, 617-19 (1968) Kamoun, J. et al. Biochemical characterization of Yarrowia lipolytica LIP8, a secreted lipase with a cleavable C-terminal region. Biochim. Biophys. Acta 1851, 129-140 (2015)
Aloulou, A. et al. Purification and biochemical characterization of the LIP2 lipase from Yarrowia lipolytica. Biochim. Biophys. Acta-Mol. Cell Biol. Lipids 1771, 228-237 (2007)
Sheng, J., Wang, F., Wang, H. & Sun, M. Cloning, characterization and expression of a novel lipase gene from marine psychrotrophic Yarrowia lipolytica. Ann. Microbiol. 62, 1071-1077 (2012)
J. Sambrook, E. F. Fritsch, and T. Maniatis, 1989, Molecular Cloning: A Laboratory manual, 2^{nd} edition, books 1-3, Cold Spring Harbor Laboratory Press
Jong C., de and Badings H. T. Determination of free fatty acids in milk and cheese procedures for extraction, clean up, and capillary gas chromatographic analysis J. High Resolution Chromatography, 13, 84-98 (1990)

### PATENT LITERATURE

U.S. Pat. Nos. 4,726,954; 4,636,468; US2005059130; EP2290059; EP3081644; EP2254996; EP1776455

SEQUENCE LISTING
>SEQ ID NO: 1
MVLSTVIGEWFSRVLFGTVAPSPLTATAPISQDFYDTALTFSHLSNVAYCINTPLESLKSDFSCGVACSHFPNMELV
EEFGGEFFETSITGFLSIDHVKKEKYVVYRGTYDIGDVYTDIQLSQSPFLVTPSALGSTANLCEGCTIHDGWNKAYN
ETMGIIGDKLADHVNSNPDYRLVVTGHSLGAAIAVLSATSLKVNGQDPYLYTYGQPRIGNANFANFVSKQWFGEGDG
LSMDSDRRYFRLTHWNDLFVGFPAFKDYVHSVGEIYIDYFTVQPPLNKVFSCAGPESMSCYRKDFNALARLDIVKNH
LAYFDWISLCTLNIGRRDLERGRKFEGTWLYGGLANGSTIF
>SEQ ID NO: 2
AVLQKRVYTSTETSHIDQESYNFFEKYARLANIGYCVGPGTKIFKPFNCGLQCAHFPNVELIEEFHDPRLIFDVSGY
LAVDHASKQIYLVIRGTHSLEDVITDIRIMQAPLTNFDLAANISSTATCDDCLVHNGFIQSYNNTYNQIGPKLDSVI
EQYPDYQIAVTGHSLGGAAALLFGINLKVNDHDPLVVTLGQPIVGNAGFANWVDKLFFGQENPDVSKVSKDRKLYRI
THRGDIVPQVPFWDGYQHCSGEVFIDWPLIHPPLSNVVMCQGQSNKQCSAGNTLLQQVNVIGNHLQYFVTEGVCGI
>SEQ ID NO: 3
QTAPITQETYDFVLKYGWLSNVAYCVRAPGPFALQSDFTCGNSCAHFPDVTLDYQFGGNFFSTSVTGFLAHDHTKKE
KYIVFRGTFSIADAITDIQTIQQPYMTSIPPLNTTDINSTNPSASINCPGCQVHDGFQKAYRETMVNVQDRLVDFLG
NNTDYKLIVTGHSLGAVTALFMGINLKNLGYDPTLINYGQPRLGNKAFADYVDALFFKQGDDGLTINPERRMYRVTH
WNDFFVGWPAGYSHTLGEVYISDPTGINAPIEDVYSCAGPENNQCHHGSFNLLERLNILKNHCGYLNWIFYCAINVD
KREMMIDPPRVGKRVEHWSGKFSDVESTEGLMYEAIYPM
>SEQ ID NO: 4
APAPAPMQRRDISSTVLDNIDLFAQYSAAAYCSSNIESTGTTLTCDVGNCPLVEAAGATTIDEFDDSSSYGDPTGFI
AVDPTNELIVLSFRGSSDLSNWIADLDFGLTSVSSICDGCEMHKGFYEAWEVIADTITSKVEAAVSSYPDYTLVFTG
HSYGAALAAVAATVLRNAGYTLDLYNFGQPRIGNLALADYITDQNMGSNYRVTHTDDIVPKLPPELLGYHHFSPEYW
ITSGNDVTVTTSDVTEVVGVDSTAGNDGTLLDSTTAHRWYTIYISECS
>SEQ ID NO: 5
VPMLQSRATSDPAEWTELHRAAQLSSAAYTGCTGSAFDVTITKQINELVTDTQGFIGYSTEKKRITVAMRGSTSATD
IANDVDTTLVEPTLSGVNFPSGAKMMHGIYSPWSSVHDDVISEVKSLVEQYPDYTIESTGHSLGGSLTYISYIALAQ
NFPGKTIISNALAAYPIGNEAFANFGASQNGTLNRGNNADDGVPNMYVMWPWDFVHYGTEYYSSGTQASTVKCSGER
DTSCSAGNGQVGVTAGHFSNFGIAMGMAGC
>SEQ ID NO: 6
APASNDATIADVSSTFTLPPIIKDRVAFSNDIADTDYFNLTKRASETVGGNTMDLPSNAPALPAVPKAGDVVIATAA
QIAEYKKYAALASTAYCRSVVPLNLWTCVNCLRFAPDGKLIKTFSSVISDTNGFVLRSDAQKTIYVVFRGTNSIRSA
ITDLVFTLISYPPVSGARVHTGFYASYQAVVSDYFPVVQSQLTSYPSYKVVVTGHSLGGAHALLAGMDLYQREKRLT
ASNLFIHTAGCPRVGNPTFANYVASTGITFTRSVNQRDIVPHVPPTYAGYLHPGVEVWARTSSTVQICTSNTESNMC
SNSIEPFTSFTDHLSYYGITEGVCI
>SEQ ID NO: 7
SPVQLARRAISSELLERFTLFSQFATLSACDQNINHTGQSLTCDYGTCGLVAADNTTVINAFHSDNGPTGYIALDHT
RQLIVLTFRGTVSKSDGDTDLDIVLTSIDDVCTGCKAHHGFWVYWSAVASQATTQLQDATSAYPSYRLSVVGHSLGG
GIAALAGTVLRTQGFTLDIWTFGGPKPGNLKLAEFITNQQPPNSIYRATHTTDPIPKVPLNLPFLDWSQPSPEYWIT
QETGVQVTTDGVEYIEGINSKAGNAGSDRDLRWPNPEHGWYFGNMSVCASPSDASS
>SEQ ID NO: 8
APSQLVPRAVSSGTLDQLTLFAQYSAASYCANNVNSPGDAITCSGGYCPKLQSAGVKSLFEFDDSTEFGDVAGFLSV
DTANKLLVLSFRGSRTISNWIANLDFGQADASSLCIGCKAHSGFLKAWTVVSDDVMPPLVSAMAKYPGFRLVLTGHS
FGGAVAALGATALRKAGYKLDLYTYGQPRVGNTALATYMTNQGSMYRVTHSNDIVPKLPPPLLGYTHASPEYWITSG
DNVAVTTKDITQVNGIGSKDGNAGSNGDSIPAHNWYIVNIDGCK
>SEQ ID NO: 9
APQKRSVSSTVLAQLSRYAQWSAAAYCSGNTSGANQVVSCSANNCPDVQASGATMLYEFDSTNTYGDAAGFLAADTT
QQQIILSFRGSRTTSNWIANLDTELTSSTLCSGCEVHQGFWLDYQTVAATLKAQIDAALNTYPGYSLVVTGHSLGGA
LAMLAGLDLNSQGYAPTIYTYGQPRVGNLALAQYITNVGNQWRVTHADDAVPKLPPRLFGFSHASPEYWITSGDNVA
VTTGDVTVVTGVNSLGGNDGTLTASVSAHNWYLVDIDACK
>SEQ ID NO: 10
APALPLEVRDSGVSQAVYDDLVVYAKYSSAVYQPFCPRPLGSWMIKAFDTKGTQGFVARDDARKEIIVAFRGSFEIV
DILIDIQIILTPLSTPGVSNVGSARVHTGFQKAYNFVIDEVQSLMKSQIDAYPSYKLVVTGHSLGGAVATFAALALK
SKYPSKSLRLFTYGAPRVGDAAFASLVESRLGINNIYRGVHTWDGVPTLLGRWLGYRHYGTEYWQHKDPSKPENVRK
CNGGEDTSCSNSIISTGINPPHAFQFGQVMAINPLLCF
>SEQ ID NO: 11
MVSFGARIKDFFSVLLFGAASTSSSTKTALVSQGFYDAALDFSHLSNIAYCVNAPITPLKSDFSCGQSCVHFPDIEL
VHIFGGDFFSTSITGYLALDHVKKEKYVVFRGTFSIADAITDIQFQQSSFLVNVPALNTFTANDTAPEAQIDCKQCK
IHDGFSKAFTETWHNIGDLLEQHLDSYPDYQLYVTGHSLGAAMALLGATSIKLRGYDPILINYGQPRVGNKAFADYI
SALWFGNGDGLEINQQRRLYRMTHWNDVFVGLPNWDGYTHSNGEVYIKGKYVNPPLKDVFSCAGGENSKCYRSEFNL
LAQINLLQNHLCYIDYIGFCALNVGRRELNDLPHYNGPYKYGHKTEEQFIAEGLELSN
>SEQ ID NO: 12
ATGGTTTTGTCTACTGTTATTGGTGAATGGTTCTCTAGAGTTTTGTTTGGTACTGTTGCTCCATCTCCTTTGACTGC
TACTGCTCCAATCTCTCAAGATTTCTACGATACTGCTTTGACTTTTTCTCATTTGTCTAACGTTGCTTACTGTATCA
ACACTCCATTGGAATCTTTGAAGTCTGATTTCTCTTGTGGTGTTGCTTGTTCTCACTTTCCTAACATGGAGTTGGTT
GAAGAGTTCGGTGGTGAATTTTTCGAGACTTCTATTACTGGTTTCTTGTCTATCGATCATGTTAAGAAAGAGAAGTA
CGTTGTTTACAGAGGTACTTACGATATCGGAGATGTTTACACTGATATCCAATTGTCTCAATCTCCATTCTTGGTTA
CTCCTTCTGCTTTGGGTTCTACTGCTAATTTGTGTGAAGGTTGTACTATTCACGATGGTTGGAACAAGGCTTATAAT
GAGACTATGGGTATTATTGGAGATAAATTGGCTGATCATGTTAACTCTAATCCTGATTACAGATTGGTTGTTACTGG
TCACTCTTTGGGTGCTGCTATTGCTGTTTTGTCTGCTACTTCTTTGAAGGTTAACGGTCAAGATCCATACTTGTATA
CTTACGGTCAACCTAGAATCGGTAACGCTAACTTCGCTAACTTCGTTTCTAAGCAATGGTTTGGTGAAGGAGATGGT
TTGTCTATGGATTCTGATAGAAGATACTTCAGATTGACTCATTGGAACGATTTGTTTGTTGGTTTCCCAGCTTTTAA
GGATTACGTTCACTCTGTTGGTGAAATCTACATCGATTACTTCACTGTTCAACCACCTTTGAACAAGGTTTTCTCTT
GTGCTGGTCCTGAGTCTATGTCTTGTTACAGAAAGGATTTCAACGCTTTGGCTAGATTGGATATCGTTAAAAATCAT
TTGGCTTACTTCGATTGGATTTCTTTGTGTACTTTGAACATTGGTAGAAGAGATTTGGAAAGAGGTAGAAAGTTTGA
GGGTACTTGGTTGTATGGTGGTTTGGCTAATGGTTCTACTATTTTC
>SEQ ID NO: 13
GCTGTTTTGCAAAAGAGAGTTTACACTTCTACTGAAACTTCTCATATCGATCAAGAATCTTACAACTTTTTCGAGAA
GTATGCTAGATTGGCTAATATTGGTTATTGTGTTGGTCCAGGTACTAAGATTTTTAAGCCTTTCAACTGTGGTTTGC
AATGTGCTCATTTCCCAAACGTTGAATTGATCGAAGAGTTTCACGATCCTAGATTGATTTTCGATGTTTCTGGTTAC
TTGGCTGTTGATCATGCTTCTAAGCAAATCTACTTGGTTATTAGAGGTACTCACTCTTTGGAGGATGTTATCACTGA
TATCAGAATCATGCAAGCTCCATTGACTAACTTTGATTTGGCTGCTAATATTTCTTCTACTGCTACTTGTGATGATT
GTTTGGTTCACAACGGTTTCATTCAATCTTACAACAACACTTACAATCAAATTGGTCCAAAGTTGGATTCTGTTATT
GAGCAATACCCTGATTATCAAATTGCTGTTACTGGTCATTCTTTGGGTGGTGCTGCTGCTTTGTTGTTCGGTATTAA
CTTGAAGGTTAATGATCACGATCCATTGGTTGTTACTTTGGGTCAACCTATTGTTGGTAACGCTGGTTTCGCTAATT
GGGTTGATAAGTTGTTTTTCGGTCAAGAAAACCCAGATGTTTCTAAGGTTTCTAAGGATAGAAAGTTGTACAGAATC
ACTCATAGAGGAGATATTGTTCCACAAGTTCCTTTTTGGGATGGTTATCAACATTGTTCTGGAGAGGTTTTCATTGA
TTGGCCTTTGATTCACCCACCTTTGTCTAATGTTGTTATGTGTCAAGGTCAATCTAACAAACAATGTTCTGCTGGTA
ACACTTTGTTGCAACAAGTTAACGTTATTGGTAATCACTTGCAATACTTCGTTACTGAAGGTGTTTGTGGTATT
>SEQ ID NO: 14
CAAACTGCTCCAATCACTCAAGAAACTTACGATTTCGTTTTGAAGTACGGTTGGTTGTCTAACGTTGCTTACTGTGT
TAGAGCTCCAGGTCCTTTTGCTTTGCAATCTGATTTCACTTGTGGTAACTCTTGTGCTCATTTCCCTGATGTTACTT
TGGATTACCAATTCGGTGGTAACTTTTTCTCTACTTCTGTTACTGGTTTCTTGGCTCACGATCACACTAAGAAAGAA
AAGTACATCGTTTTTAGAGGTACTTTCTCTATCGCTGATGCTATTACTGATATCCAAACTATCCAACAACCATACAT
GACTTCTATTCCACCTTTGAACACTACTGATATCAACTCTACTAATCCATCTGCTTCTATTAATTGTCCTGGTTGTC
AAGTTCACGATGGTTTTCAAAAAGCTTACAGAGAGACTATGGTTAACGTTCAAGATAGATTGGTTGATTTCTTGGGT
AACAACACTGATTACAAGTTGATCGTTACTGGTCACTCTTTGGGTGCTGTTACTGCTTTGTTTATGGGTATTAACTT
GAAAAATTTGGGTTACGATCCAACTTTGATTAACTATGGTCAACCTAGATTGGGTAATAAGGCTTTCGCTGATTACG
TTGATGCTTTGTTTTTCAAACAAGGAGATGATGGTTTGACTATTAACCCAGAAAGAAGAATGTACAGAGTTACTCAT
TGGAACGATTTCTTTGTTGGTTGGCCTGCTGGTTACTCTCACACTTTGGGTGAAGTTTATATTTCTGACCCAACTGG
TATTAACGCTCCTATTGAAGATGTTTACTCTTGTGCTGGTCCAGAGAACAATCAATGTCATCACGGTTCTTTTAATT
TGTTGGAAAGATTGAACATCTTGAAGAATCATTGTGGTTACTTGAACTGGATTTTCTATTGTGCTATTAATGTTGAT
AAGAGAGAAATGATGATTGATCCACCTAGAGTTGGTAAAAGAGTTGAGCACTGGTCTGGTAAATTTTCTGATGTTGA
ATCTACTGAGGGTTTGATGTACGAGGCTATCTACCCTATG
>SEQ ID NO: 15
GCTCCAGCTCCTGCTCCAATGCAAAGAAGAGATATCTCTTCTACTGTTTTGGATAACATCGATTTGTTCGCTCAATA
CTCTGCTGCTGCTTATTGTTCTTCTAACATTGAATCTACTGGTACTACTTTGACTTGTGATGTTGGTAATTGTCCTT
TGGTTGAAGCTGCTGGTGCTACTACTATCGATGAGTTCGATGATTCTTCTTCTTACGGAGATCCTACTGGTTTCATT
GCTGTTGATCCAACTAACGAGTTGATTGTTTTGTCTTTTAGAGGTTCTTCTGATTTGTCTAATTGGATTGCTGATTT
GGATTTCGGTTTGACTTCTGTTTCTTCTATTTGTGATGGTTGTGAAATGCATAAGGGTTTTTACGAAGCTTGGGAGG
TTATTGCTGATACTATCACTTCTAAGGTTGAGGCTGCTGTTTCTTCTTACCCTGATTATACTTTGGTTTTCACTGGT
CACTCTTATGGTGCTGCTTTGGCTGCTGTTGCTGCTACTGTTTTGAGAAATGCTGGTTACACTTTGGATTTGTATAA
CTTTGGTCAACCAAGAATTGGTAATTTGGCTTTGGCTGATTACATCACTGATCAAAACATGGGTTCTAACTACAGAG
TTACTCATACTGATGATATTGTTCCTAAGTTGCCACCTGAATTGTTGGGTTACCATCACTTTTCTCCAGAGTATTGG
ATTACTTCTGGTAACGATGTTACTGTTACTACTTCTGATGTTACTGAAGTTGTTGGTGTTGATTCTACTGCTGGTAA
TGATGGTACTTTGTTGGATTCTACTACTGCTCACAGATGGTACACTATCTACATTTCTGAGTGTTCT
>SEQ ID NO: 16
GTTCCAATGTTGCAATCTAGAGCTACTTCTGATCCTGCTGAATGGACTGAGTTGCATAGAGCTGCTCAATTGTCTTC
TGCTGCTTACACTGGTTGTACTGGTTCTGCTTTCGATGTTACTATCACTAAGCAAATTAACGAATTGGTTACTGATA
CTCAAGGTTTCATTGGTTACTCTACTGAGAAGAAAAGAATCACTGTTGCTATGAGAGGTTCTACTTCTGCTACTGAT
ATTGCTAACGATGTTGATACTACTTTGGTTGAACCAACTTTGTCTGGTGTTAATTTTCCTTCTGGTGCTAAGATGAT
GCATGGTATCTACTCTCCATGGTCTTCTGTTCACGATGATGTTATCTCTGAGGTTAAGTCTTTGGTTGAGCAATACC
CTGATTATACTATTGAATCTACTGGTCACTCTTTGGGTGGTTCTTTGACTTACATCTCTTACATCGCTTTGGCTCAA
AACTTCCCAGGTAAAACTATCATCTCTAACGCTTTGGCTGCTTATCCTATTGGTAACGAGGCTTTTGCTAATTTCGG
TGCTTCTCAAAACGGTACTTTGAATCGTGGTAACAATGCTGATGATGGTGTTCCAAATATGTACGTTATGTGGCCTT
GGGATTTCGTTCATTACGGTACTGAATACTACTCTTCTGGTACTCAAGCTTCTACTGTTAAATGTTCTGGAGAGAGA
GATACTTCTTGTTCTGCTGGTAACGGTCAAGTTGGTGTTACTGCTGGTCACTTTTCTAATTTCGGTATTGCTATGGG
TATGGCTGGTTGT
>SEQ ID NO: 17
GCTCCTGCTTCTAATGATGCTACTATTGCTGATGTTTCTTCTACTTTCACTTTGCCACCTATTATTAAGGATAGAGT
TGCTTTTTCTAACGATATCGCTGATACTGATTACTTCAACTTGACTAAGAGAGCTTCTGAAACTGTTGGTGGTAATA
CTATGGATTTGCCATCTAACGCTCCAGCTTTGCCTGCTGTTCCAAAGGCTGGAGATGTTGTTATTGCTACTGCTGCT
CAAATTGCTGAGTACAAGAAATATGCTGCTTTGGCTTCTACTGCTTACTGTAGATCTGTTGTTCCTTTGAATTTGTG
GACTTGTGTTAACTGTTTGAGATTTGCTCCAGATGGTAAATTGATTAAAACTTTCTCTTCTGTTATTTCTGATACTA
ACGGTTTCGTTTTGAGATCTGATGCTCAAAAGACTATCTACGTTGTTTTCAGAGGTACTAACTCTATCAGATCTGCT
ATCACTGATTTGGTTTTTACTTTGATCTCTTACCCACCTGTTTCTGGTGCTAGAGTTCATACTGGTTTCTACGCTTC
TTATCAAGCTGTTGTTTCTGATTACTTTCCTGTTGTTCAATCTCAATTGACTTCTTACCCATCTTATAAGGTTGTTG
TTACTGGTCATTCTTTGGGTGGTGCTCACGCTTTGTTGGCTGGTATGGATTTGTACCAAAGAGAAAAGAGATTGACT
GCTTCTAATTTGTTTATTCACACTGCTGGTTGTCCTAGAGTTGGTAATCCAACTTTCGCTAACTACGTTGCTTCTAC
TGGTATCACTTTTACTAGATCTGTTAACCAAAGAGATATTGTTCCTCATGTTCCACCTACTTACGCTGGTTATTTGC
ACCCAGGTGTTGAAGTTTGGGCTAGAACTTCTTCTACTGTTCAAATCTGTACTTCTAACACTGAATCTAACATGTGT
TCTAACTCTATCGAGCCTTTTACTTCTTTCACTGATCATTTGTCTTACTATGGTATTACTGAGGGTGTTTGTATT
>SEQ ID NO: 18
TCTCCAGTTCAATTGGCTAGAAGAGCTATTTCTTCTGAATTGTTGGAGAGATTCACTTTGTTCTCTCAATTTGCTAC
TTTGTCTGCTTGTGATCAAAACATCAACCATACTGGTCAATCTTTGACTTGTGATTACGGTACTTGTGGTTTGGTTG
CTGCTGATAACACTACTGTTATTAATGCTTTCCATTCTGATAACGGTCCTACTGGTTATATTGCTTTGGATCACACT
AGACAATTGATCGTTTTGACTTTTAGAGGTACTGTTTCTAAGTCTGATGGAGATACTGATTTGGATATCGTTTTGAC
TTCTATCGATGATGTTTGTACTGGTTGTAAAGCTCATCACGGTTTCTGGGTTTACTGGTCTGCTGTTGCTTCTCAAG
CTACTACTCAATTGCAAGATGCTACTTCTGCTTACCCATCTTATAGATTGTCTGTTGTTGGTCATTCTTTGGGTGGT
GGTATTGCTGCTTTGGCTGGTACTGTTTTGAGAACTCAAGGTTTCACTTTGGATATTTGGACTTTTGGTGGTCCAAA
GCCTGGTAACTTGAAGTTGGCTGAATTCATTACTAACCAACAACCACCTAATTCTATCTACAGAGCTACTCACACTA
CTGATCCAATTCCTAAGGTTCCATTGAATTTGCCATTTTTGGATTGGTCTCAACCATCTCCTGAATACTGGATTACT
CAAGAGACTGGTGTTCAAGTTACTACTGATGGTGTTGAATACATCGAGGGTATTAACTCTAAAGCTGGTAATGCTGG
TTCTGATAGAGATTTGAGATGGCCAAACCCTGAGCACGGTTGGTATTTTGGTAATATGTCTGTTTGTGCTTCTCCTT
CTGATGCTTCTTCT
>SEQ ID NO: 19
GCTCCTTCTCAATTGGTTCCAAGAGCTGTTTCTTCTGGTACTTTGGATCAATTGACTTTGTTTGCTCAATACTCTGC
TGCTTCTTATTGTGCTAACAATGTTAACTCTCCTGGAGATGCTATTACTTGTTCTGGTGGTTACTGTCCAAAGTTGC
AATCTGCTGGTGTTAAGTCTTTGTTCGAATTCGATGATTCTACTGAGTTTGGAGATGTTGCTGGTTTCTTGTCTGTT
GATACTGCTAATAAGTTGTTGGTTTTGTCTTTTAGAGGTTCTAGAACTATCTCTAACTGGATCGCTAATTTGGATTT
CGGTCAAGCTGATGCTTCTTCTTTGTGTATTGGTTGTAAGGCTCATTCTGGTTTCTTGAAAGCTTGGACTGTTGTTT
CTGATGATGTTATGCCACCTTTGGTTTCTGCTATGGCTAAATACCCTGGTTTTAGATTGGTTTTGACTGGTCACTCT
TTCGGTGGTGCTGTTGCTGCTTTGGGTGCTACTGCTTTGAGAAAGGCTGGTTACAAGTTGGATTTGTACACTTACGG
TCAACCAAGAGTTGGTAACACTGCTTTGGCTACTTACATGACTAATCAAGGTTCTATGTACAGAGTTACTCATTCTA
ACGATATCGTTCCTAAGTTGCCACCTCCATTGTTGGGTTACACTCACGCTTCTCCAGAATATTGGATTACTTCTGGA
GATAACGTTGCTGTTACTACTAAGGATATCACTCAAGTTAACGGTATCGGTTCTAAAGATGGTAACGCTGGTTCTAA
TGGAGATTCTATTCCTGCTCATAACTGGTATATTGTTAATATTGATGGTTGTAAA
>SEQ ID NO: 20
GCTCCACAAAAGAGATCTGTTTCTTCTACTGTTTTGGCTCAATTGTCTAGATACGCTCAATGGTCTGCTGCTGCTTA
TTGTTCTGGTAACACTTCTGGTGCTAATCAAGTTGTTTCTTGTTCTGCTAACAATTGTCCTGATGTTCAAGCTTCTG
GTGCTACTATGTTGTACGAATTCGATTCTACTAACACTTACGGAGATGCTGCTGGTTTCTTGGCTGCTGATACTACT
CAACAACAAATCATCTTGTCTTTTAGAGGTTCTAGAACTACTTCTAACTGGATTGCTAATTTGGATACTGAATTGAC
TTCTTCTACTTTGTGTTCTGGTTGTGAGGTTCATCAAGGTTTCTGGTTGGATTACCAAACTGTTGCTGCTACTTTGA
AAGCTCAAATTGATGCTGCTTTGAATACTTACCCAGGTTATTCTTTGGTTGTTACTGGTCACTCTTTGGGTGGTGCT
TTGGCTATGTTGGCTGGTTTGGATTTGAACTCTCAAGGTTACGCTCCAACTATCTACACTTATGGTCAACCTAGAGT
TGGTAATTTGGCTTTGGCTCAATACATCACTAACGTTGGTAACCAATGGAGAGTTACTCATGCTGATGATGCTGTTC
CAAAGTTGCCACCTAGATTGTTTGGTTTCTCTCACGCTTCTCCTGAGTACTGGATTACTTCTGGAGATAACGTTGCT
GTTACTACTGGAGATGTTACTGTTGTTACTGGTGTTAACTCTTTGGGTGGTAATGATGGTACTTTGACTGCTTCTGT
TTCTGCTCATAACTGGTATTTGGTTGATATCGATGCTTGTAAA
>SEQ ID NO: 21
GCTCCAGCTTTGCCTTTGGAAGTTAGAGATTCTGGTGTTTCTCAAGCTGTTTACGATGATTTGGTTGTTTACGCTAA
GTATTCTTCTGCTGTTTATCAACCATTTTGTCCAAGACCTTTGGGTTCTTGGATGATTAAGGCTTTTGATACTAAAG
GTACTCAAGGTTTCGTTGCTAGAGATGATGCTAGAAAGGAAATCATCGTTGCTTTTAGAGGTTCTTTCGAGATTGTT
GATATCTTGATCGATATCCAAATCATCTTGACTCCATTGTCTACTCCTGGTGTTTCTAACGTTGGTTCTGCTAGAGT
TCATACTGGTTTCCAAAAGGCTTACAACTTCGTTATCGATGAGGTTCAATCTTTGATGAAGTCTCAAATCGATGCTT
ACCCTTCTTACAAGTTGGTTGTTACTGGTCACTCTTTGGGTGGTGCTGTTGCTACTTTCGCTGCTTTGGCTTTGAAG
TCTAAGTACCCATCTAAGTCTTTGAGATTGTTTACTTACGGTGCTCCTAGAGTTGGAGATGCTGCTTTCGCTTCTTT
GGTTGAATCTAGATTGGGTATTAACAACATCTACAGAGGTGTTCATACTTGGGATGGTGTTCCAACTTTGTTGGGTA
GATGGTTGGGTTACAGACATTATGGTACTGAGTATTGGCAACACAAGGATCCATCTAAACCTGAAAACGTTAGAAAG
TGTAATGGTGGAGAGGATACTTCTTGTTCTAACTCTATCATCTCTACTGGTATTAATCCACCTCACGCTTTTCAATT
CGGTCAAGTTATGGCTATTAACCCTTTGTTGTGTTTT
>SEQ ID NO: 22
ATGGTTTCTTTCGGTGCTAGAATTAAGGATTTCTTTTCTGTTTTGTTGTTTGGTGCTGCTTCTACTTCTTCTTCTAC
TAAAACTGCTTTGGTTTCTCAAGGTTTTTATGATGCTGCTTTGGATTTCTCTCATTTGTCTAACATCGCTTACTGTG
TTAACGCTCCAATTACTCCTTTGAAGTCTGATTTTTCTTGTGGTCAATCTTGTGTTCATTTCCCAGATATTGAATTG
GTTCACATTTTTGGTGGAGATTTCTTTTCTACTTCTATCACTGGTTATTTGGCTTTGGATCACGTTAAGAAAGAGAA
GTACGTTGTTTTTAGAGGTACTTTCTCTATCGCTGATGCTATCACTGATATCCAATTCCAACAATCTTCTTTCTTGG
TTAACGTTCCAGCTTTGAATACTTTCACTGCTAACGATACTGCTCCTGAAGCTCAAATCGATTGTAAGCAGTGTAAG
ATTCACGATGGTTTTTCTAAGGCTTTCACTGAAACTTGGCATAACATTGGAGATTTGTTGGAGCAACACTTGGATTC
TTACCCAGATTATCAATTGTACGTTACTGGTCACTCTTTGGGTGCTGCTATGGCTTTGTTGGGTGCTACTTCTATTA
AGTTGAGAGGTTACGATCCAATCTTGATTAATTACGGTCAACCTAGAGTTGGTAACAAAGCTTTCGCTGATTATATT
TCTGCTTTGTGGTTTGGTAATGGAGATGGTTTGGAAATTAACCAACAAAGAAGATTGTACAGAATGACTCATTGGAA
CGATGTTTTCGTTGGTTTGCCTAACTGGGATGGTTACACTCACTCTAACGGAGAGGTTTACATCAAGGGTAAATACG
TTAACCCACCTTTGAAGGATGTTTTCTCTTGTGCTGGTGGTGAAAACTCTAAGTGTTACAGATCTGAGTTTAATTTG
TTGGCTCAAATTAACTTGTTGCAAAACCATTTGTGTTACATCGATTACATCGGTTTCTGTGCTTTGAACGTTGGTAG
AAGAGAGTTGAATGATTTGCCACATTACAACGGTCCTTACAAGTATGGTCACAAAACTGAAGAGCAATTCATTGCTG
AAGGTTTGGAGTTGTCTAAT
>SEQ ID NO: 23
ATGGTCCTGTCCACTGTTATCGGAGAATGGTTTTCAAGAGTTTTGTTTGGTACTGTCGCCCCATCACCACTCACCGC
CACAGCCCCCATTTCTCAGGACTTCTACGATACCGCTCTCACGTTCTCTCACCTATCTAATGTTGCTTACTGTATCA
ACACCCCTCTTGAGTCCCTCAAGTCCGACTTTTCCTGCGGTGTCGCATGTTCTCACTTCCCAAACATGGAACTTGTT
GAAGAGTTCGGTGGAGAATTTTTCGAAACTTCTATTACTGGTTTTCTATCTATCGACCATGTCAAAAAGGAAAAGTA
CGTTGTGTACAGAGGCACCTATGACATTGGTGACGTTTATACTGACATTCAATTAAGCCAATCCCCATTCTTGGTTA
CCCCTTCTGCCCTGGGTTCTACAGCAAATCTGTGTGAGGGTTGTACTATCCACGATGGTTGGAACAAAGCATACAAC
GAAACCATGGGTATTATCGGAGACAAGCTCGCTGACCACGTTAACTCCAACCCAGATTACAGATTGGTCGTAACTGG
ACATTCACTTGGTGCTGCTATTGCTGTACTATCTGCAACCTCTCTTAAGGTCAATGGCCAAGATCCTTACCTTTACA
CTTACGGTCAGCCTCGTATTGGTAATGCTAACTTCGCAAACTTTGTGAGTAAGCAATGGTTCGGTGAGGGTGACGGT
CTATCTATGGACTCGGACAGACGTTATTTCAGACTGACTCACTGGAACGACCTCTTTGTTGGTTTCCCTGCTTTCAA
GGACTACGTGCACTCTGTCGGAGAGATTTACATAGACTATTTCACCGTTCAACCACCACTCAACAAAGTTTTCTCTT
GTGCTGGGCCTGAGTCTATGTCCTGTTACAGAAAGGACTTCAACGCCCTTGCTAGACTTGATATTGTGAAAAACCAC
CTAGCTTACTTCGATTGGATCTCTCTGTGCACCCTAAACATCGGCAGACGTGATCTGGAGAGGGGTAGAAAGTTCGA
GGGTACTTGGCTCTACGGTGGACTGGCTAACGGATCCACTATCTTC

1-15. (canceled)

16. A method for preparing a food product, comprising adding to a raw material or intermediate product of the food product a lipase or host cell transformed to express a DNA sequence encoding a lipase,
wherein the lipase is selected from:
(i) lipases comprising the amino acid sequence of any one of SEQ ID NOs. 1-11, and
(ii) lipases comprising an amino acid sequence having an identity to any one of SEQ ID NOs. 1-11 selected from at least 90%, at least 95%, at least 96%, at least 97%, at least 98%, and at least 99%;
wherein the DNA sequence encoding the lipase is selected from:
(i) DNA sequences comprising the nucleotide sequence of any one of SEQ ID NOs. 12-23, and
(ii) DNA sequences comprising a nucleotide sequence having an identity to any one of SEQ ID NOs. 12-23 selected from at least 90%, at least 95%, at least 96%, at least 97%, at least 98%, and at least 99%.

17. The method of claim 16, wherein the method comprises adding a host cell transformed to express the DNA sequence encoding the lipase, wherein the DNA sequence is selected from isolated DNA sequences, recombinant DNA sequences, and synthetic DNA sequences.

18. The method of claim 16, wherein the method comprises adding a host cell transformed to express the DNA sequence encoding the lipase, wherein the DNA sequence further comprises a nucleotide sequence encoding a signal peptide.

19. The method of claim 16, wherein the lipase has a higher specificity towards release of C₄-fatty acids as compared to release of one or more of C₁₀-fatty acids, C₈-fatty acids, C₆-fatty acids, C₁₂-fatty acids, C_{18:2}-fatty acids, C₁₄-fatty acids, C_{18:0}-fatty acids, C_{18:1}-fatty acids, C₈-fatty acids-C_{18:2}-fatty acids, C₁₀-fatty acids-C_{18:1}-fatty acids, C₁₂-fatty acids-C_{18:0}-fatty acids, C₁₄-fatty acids-C_{16:0}-fatty acids, and C₁₆-fatty acids-C₁₈-fatty acids, when assessed in a dairy composition comprising milk fat.

20. The method of claim 16, wherein the lipase has a higher specificity towards release of C₄-fatty acids as compared to release of one or more of C₁₀-fatty acids, C₆-fatty acids, C₈-fatty acids, C₁₂-fatty acids, C₁₄-fatty acids, C₁₆-fatty acids, C_{18:1}-fatty acids, C_{18:2}-fatty acids, C_{18:3}-fatty acids, and C₁₆-C₁₈-fatty acids, when assessed in a dairy composition comprising milk fat at a pH selected from a pH below 7, a pH of 6.6-6.8, a pH below 6, a pH of 3.8-5.6, a pH of 4.4-5.4, and a pH of 4.6-5.2.

21. The method of claim 16, wherein the lipase has a higher specificity towards release of C₄-fatty acids as compared to release of one or more of C₁₀-fatty acids, C₆-fatty acids, C₈-fatty acids, C₁₂-fatty acids, C₁₄-fatty acids, C₁₆-fatty acids, C_{18:1}-fatty acids, C_{18:2}-fatty acids, C_{18:3}-fatty acids, and C₁₆-C₁₈-fatty acids, when assessed in a dairy composition comprising milk fat at a selected from a temperature below 20 °C, a temperature below 15 °C, a temperature below 10 °C, and a temperature of 5-8 °C.

22. The method of claim 16, wherein the food product obtained by the method has one or more fatty acid profiles selected from:
(a) comprising more C₄-fatty acids than one or more other fatty acids selected from C₆-fatty acids, C₈-fatty acids, C₁₀-fatty acids, C₁₂-fatty acids, C₁₄-fatty acids, C₁₆-fatty acids, C_{18:1}-fatty acids, C_{18:2}-fatty acids and C_{18:3}-fatty acids, and
(b) comprising more C₆-fatty acids than one or more other fatty acids selected from C₈-fatty acids, C₁₀-fatty acids, C₁₂-fatty acids, C₁₄-fatty acids, C₁₆-fatty acids, C_{18:1}-fatty acids, C_{18:2}-fatty acids and C_{18:3}-fatty acids.

23. The method of claim 16, wherein the food product has one or more flavor profiles selected from reduced soapiness and increased butyric flavors, as compared to a food product made without the lipase and without the host cell transformed to express a a DNA sequence encoding the lipase.

24. The method of claim 16, wherein the lipase is a microbial lipase selected from an isolated microbial lipase, a recombinant microbial lipase, and a synthetic microbial lipase.

25. The method of claim 16, wherein the food product is a dairy product selected from a cheese, a processed cheese, a cheese-like product, an enzyme-modified cheese, a butter, a yogurt, a cream, and a seasoning.

26. The method of claim 16, wherein the food product is a cheese selected from Feta cheese, Provolone cheese, Pecorino cheese, Parmesan cheese, Grana Padano cheese, Parmigiano Reggiano cheese, Romano cheese, Chester cheese, Danbo cheese, Manchego cheese, Saint Paulin cheese, Cheddar cheese, Monterey cheese, Colby cheese, Edam cheese, Gouda cheese, Muenster cheese, Swiss-type cheese, Gruyere cheese, Emmental cheese; Mozzarella cheese, Queso fresco cheese, Ricotta cheese, Cream cheese, Neufchatel cheese, Cottage cheese, Brie, Camembert, Gorgonzola and Danish blue cheese.

27. The method of claim 16, wherein the food product is a non-dairy food product.

28. A food product comprising a lipase, wherein the lipase is selected from:
(i) lipases comprising the amino acid sequence of any one of SEQ ID NOs. 1-11, and
(ii) lipases comprising an amino acid sequence having an identity to any one of SEQ ID NOs. 1-11 selected from at least 90%, at least 95%, at least 96%, at least 97%, at least 98%, and at least 99%;
wherein the food product has one or more fatty acid profiles selected from:
(a) a fatty acid profile comprising more C₄-fatty acids than one or more other fatty acids selected from C₆-fatty acids, C₈-fatty acids, C₁₀-fatty acids, C₁₂-fatty acids, C₁₄-fatty acids, C₁₆-fatty acids, C_{18:1}-fatty acids, C_{18:2}-fatty acids and C_{18:3}-fatty acids, and
(b) a fatty acid profile comprising more C₆-fatty acids than one or more other fatty acids selected from C₈-fatty acids, C₁₀-fatty acids, C₁₂-fatty acids, C₁₄-fatty acids, C₁₆-fatty acids, C_{18:1}-fatty acids, C_{18:2}-fatty acids and C_{18:3}-fatty acids.

29. The food product of claim 28, wherein the food product is a dairy product selected from a cheese, a processed cheese, a cheese-like product, an enzyme-modified cheese, a butter, a yogurt, a cream, and a seasoning.

30. The food product of claim 28, wherein the food product is a cheese selected from Feta cheese, Provolone cheese, Pecorino cheese, Parmesan cheese, Grana Padano cheese, Parmigiano Reggiano cheese, Romano cheese, Chester cheese, Danbo cheese, Manchego cheese, Saint Paulin cheese, Cheddar cheese, Monterey cheese, Colby cheese, Edam cheese, Gouda cheese, Muenster cheese, Swiss-type cheese, Gruyere cheese, Emmental cheese; Mozzarella cheese, Queso fresco cheese, Ricotta cheese, Cream cheese, Neufchatel cheese, Cottage cheese, Brie, Camembert, Gorgonzola and Danish blue cheese.

31. The food product of claim 28, wherein the food product is a non-dairy food product.

32. An isolated lipase selected from:
(i) lipases comprising the amino acid sequence of any one of SEQ ID NOs. 1-11, and
(ii) lipases comprising an amino acid sequence having an identity to any one of SEQ ID NOs. 1-11 selected from at least 90%, at least 95%, at least 96%, at least 97%, at least 98%, and at least 99%,
wherein the lipase is expressed in Pichia pastoris and wherein the lipase has a higher specificity towards release of C₄-fatty acids as compared to release of one or more of C₁₀-fatty acids, C₈-fatty acids, C₆-fatty acids, C₁₂-fatty acids, C_{18:2}-fatty acids, C₁₄-fatty acids, C_{18:0}-fatty acids, C_{18:1}-fatty acids, and C₁₆-fatty acids, when assessed in a dairy composition comprising milk fat.

33. An isolated nucleic acid molecule encoding a lipase and having a DNA sequence selected from:
(i) DNA sequences comprising the nucleotide sequence of any one of SEQ ID NOs. 12-23, and
(ii) DNA sequences comprising a nucleotide sequence having an identity to any one of SEQ ID NOs. 12-23 selected from at least 90%, at least 95%, at least 96%, at least 97%, at least 98%, and at least 99%,
wherein the lipase has a higher specificity towards release of C₄-fatty acids as compared to release of one or more of C₁₀-fatty acids, C₈-fatty acids, C₆-fatty acids, C₁₂-fatty acids, C_{18:2}-fatty acids, C₁₄-fatty acids, C_{18:0}-fatty acids, C_{18:1}-fatty acids, and C₁₆-fatty acids, when assessed in a dairy composition comprising milk fat.

34. An isolated host cell engineered to express a lipase according to claim 32.

35. An isolated host cell transformed with a nucleic acid molecule according to claim 33.
